SEQUENCE LISTING (4 sequences)

SEQ ID NO: 1 - protein, 324 aa, Terribacillus saccharophilus [sequence omitted]

SEQ ID NO: 2 - protein, 324 aa, synthetic construct
  REGION 1..324: note = variant
QDIEKGITIDISRKYYTVETLKSLVDEISYNGGNYVQLHFSDNENYAIWSEYLGQSSEETNNTYLTKNELLSLIAYSNDKDILVIPDIDLPAHSKGWLELIKKKDVKLYNDIVTDYSEETLDYYDNRVALDTVNQLLDEVLDLFYQPKFEGKQRIVLGGDEVPGSEVHQLDFIDFMNQIASTVKERKYEPQMWNDSITSEGIANLDDSFSILYWQQSTLSSGEEGLTVEDFDNWWFSVYNYNAYSLYFLPSPGFTQEDIQEQMDYMNWAYAVNKFFYIDDPYHAVETSNVKGSSLTFWGEHATDLSQQELLQQELPLIRHYLNL

SEQ ID NO: 3 - protein, 324 aa, synthetic construct
  REGION 1..324: note = variant
QDFEKGITIDISRKYYTVETLKSLVDEISYNGGNYVQLHFSDNENYAIASEYLGQSSENTNNTYLTKNELLSLIAYSNDKDILVIPDIDLPAHSKGWLELIKKKDVKLYNDIVTDYSEETLDYYDNRVALDTVNQLLDEILDLFYQPKFEGKQRIVLGGDEVPGSEVHQLDFIDFMNQIASTVKERKYEPQMWNDSITSEGIANLDDSFSILYWKQSTLSSGEEGLTVEDFDNWWFSVYNYNAYSLYFLPSPGFTQEDIQEQMDYMTWAYAVNKFFYIDDPYHAVETSNVKGSSLTFWGEHATDLSQQELLQQELPLIRHYLNL

SEQ ID NO: 4 - protein, 324 aa, synthetic construct
  REGION 1..324: note = variant
QDQEKGITIDISRKHYTVETLKSLVDEISYNGGNYVQLHFSDNENYAIASEYLGQSSENTNNTYLTKNELLSLIAYSNDKDILVIPDIDLPAHSKGWLELIKKKDVKLYNDIVTDYSEETLDYYDNRVALDTVNQLLDEVLDLFYQPKFEGKQRIVLGGDEVPGSEVHQLDFIDFMNQIASTVKESKYEPQMWNDSITSEGIANLDDSFSILYWKQSTLSSGEESLTVEDFENWGFSVYNYNAYSLYFLPSPGFTQEDINEQMDYMNWAYAHNKFAYISDYYHAVETSNVKGSSLTFWGEHATDLSQEELLEQELPLIRHYLNL

(19) United States
(12) Patent Application Publication
(10) Pub. No.: US 2025/0207116 A1
(43) Pub. Date: Jun. 26, 2025

(54) HEXOSAMINIDASE VARIANTS AND COMPOSITIONS

(71) Applicant: NOVOZYMES A/S, Bagsvaerd (DK)

(72) Inventors: Lars Henrik OESTERGAARD, Charlottelund (DK); Annette Helle JOHANSEN, Bronshoj (DK); Martin GUDMAND, Holte (DK); Kaspar Damgaard TIDEMAND, Copenhagen (DK); Rolf Thomas LENHARD, Valby (DK); Roland Alexander PACHE, Allerod (DK); Rebecca Munk VEJBORG, Allerod (DK); Johanne Moerch JENSEN, Brighton (AU)

(73) Assignee: NOVOZYMES A/S, Bagsvaerd (DK)

(21) Appl. No.: 18/848,281

(22) PCT Filed: Mar. 30, 2023
(86) PCT No.: PCT/EP2023/058333

§ 371 Date: Sep. 18, 2024

(30) Foreign Application Priority Data
Apr. 8, 2022 (EP) 22167309.8

(51) Int. Cl. C12N 9/24; C11D 3/386; C12N 15/75; C12R 1/07

(52) U.S. Cl. CPC C12N 9/2402; C11D 3/38636; C12N 15/75; C11D 2111/12; C12R 2001/07; C12Y 302/01052

(57) ABSTRACT

The present invention relates to novel hexosaminidase variants exhibiting alterations relative to a parent hexosaminidase that have been found to provide improved stability, e.g. improved detergent stability. The invention also relates to detergent compositions comprising the hexosaminidase variants, and methods for using and producing the hexosaminidase variants, as well as isolated hexosaminidase sequences encoding the variants, expression vectors and host cells. The variants of the present invention are suitable for use in cleaning processes and detergent compositions.

### REFERENCE TO A SEQUENCE LISTING

This application contains a sequence listing in computer readable form, which is incorporated herein by reference.

### BACKGROUND OF THE INVENTION

### Field of the Invention

The present invention relates to novel hexosaminidase (dispersin) variants, compositions such as detergent compositions comprising the variants, polynucleotides encoding the variants, methods of producing the variants, methods of using the variants, and use of the variants and detergent compositions for cleaning.

### Description of the Related Art

Various enzymes such as proteases and amylases have long been used in detergent compositions for removing protein and starch related soiling, respectively. However, most soiling in e.g. laundry is a complex mixture of various organic matter that can be difficult to fully remove even using known enzymes. For example, complex organic stains such as biosoiling from human skin, e.g. dead skin cells, sweat and sebum, comprises different organic substances such as protein, starch, oil as well as e.g. polysaccharides. Consequently, effective stain removal in e.g. laundry requires different enzyme activities.

Detergent compositions comprising enzymes having hexosaminidase activity (dispersins) and hexosamindase enzyme variants have previously been disclosed, e.g. in WO 2017/186943 and WO 2020/207944. However, detergent compositions containing a dispersin are not yet commercially available, and further, different detergent compositions and washing conditions may present challenges that not all known dispersins are able to address.

In general, to be useful in cleaning processes such as laundry, an enzyme such as a dispersin needs to be stable and effective in a given detergent composition and under a given set of wash conditions. For example, laundry conditions in North America and certain other parts of the world typically differ from those in e.g. Europe. In Europe, washing machines are typically front-loading and use a relatively small amount of water in combination with relatively long wash cycles and often a higher temperature, whereas in North America and elsewhere, washing machines are typically top-loading and use a large amount of water, relatively short wash cycles and often lower temperatures. These conditions in e.g. North America and elsewhere, including low to medium detergent concentrations in the wash water, can make it difficult for enzymatic detergents to provide effective stain removal in the highly diluted wash water with short wash cycles and often relatively low temperature. Detergent compositions may also be formulated differently e.g. in North America compared to Europe to address the wash conditions as well as other local factors such as water hardness.

The present invention provides such hexosaminidase enzyme variants that have been found to be particularly suitable for use e.g. under typical North American washing conditions and in detergent compositions that are typically used in e.g. North America.

### SUMMARY OF THE INVENTION

The present invention relates to variants of the hexosaminidase polypeptide of SEQ ID NO: 1, wherein the variant comprises compared to SEQ ID NO: 1:
a substitution at one or more positions, preferably two, three or four positions, corresponding to positions 163, 227, 252 and 309 of SEQ ID NO: 1; and
a substitution at one or more positions corresponding to positions 106, 111, 120, 124, 127, 150, 170, 171, 178, 199, 208, 254, 255 and 278 of SEQ ID NO: 1;
wherein the variant has hexosaminidase activity and has at least 60%, e.g., at least 65%, at least 70%, at least 75%, at least 80%, at least 85%, at least 90% or at least 95% sequence identity, but less than 100% sequence identity, to the polypeptide of SEQ ID NO: 1, SEQ ID NO: 2 or SEQ ID NO: 3.

The present invention also relates to compositions such as detergent compositions comprising a hexosaminidase variant disclosed herein as well as isolated polynucleotides encoding the variants; nucleic acid constructs, vectors, and host cells comprising the polynucleotides, and methods of producing the variants. The invention further relates to methods of using the hexosaminidase variants and compositions for cleaning, e.g. for laundry.

### BRIEF DESCRIPTION OF THE FIGURE

FIG. 1 is an alignment of the polypeptides of SEQ ID NOs: 1, 2, 3 and 4.

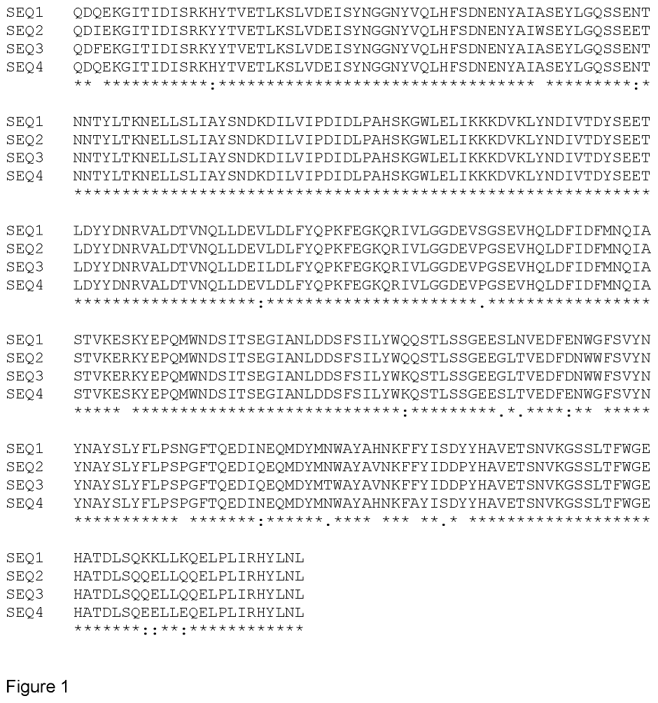

### OVERVIEW OF SEQUENCE LISTING

SEQ ID NO: 1 mature hexosaminidase polypeptide obtained from Terribacillus saccharophilus

SEQ ID NO: 2 hexosaminidase, variant of SEQ ID NO: 1

SEQ ID NO: 3 hexosaminidase, variant of SEQ ID NO: 1

SEQ ID NO: 4 hexosaminidase, variant of SEQ ID NO: 1

### Definitions

In accordance with this detailed description, the following definitions apply. Note that the singular forms “a,” “an,” and “the” include plural references unless the context clearly dictates otherwise.

Unless defied otherwise or clearly indicated by context, all technical and scientific terms used herein have the same meaning as commonly understood by one of ordinary skill in the art to which this invention belongs.

Hexosaminidase: The term “hexosaminidase” includes the term “dispersin” and the abbreviation “Dsp”, and refers to a polypeptide having hexosaminidase activity, EC 3.2.1., that catalyzes the hydrolysis of β-1,6-glycosidic linkages of N-acetyl-glucosamine polymers found in, e.g., biofilm. The term hexosaminidase includes polypeptides having N-acetylglucosaminidase activity and β-N-acetylglucosaminidase activity. The term “polypeptide having hexosaminidase activity” may be used interchangeably with the term “hexosaminidase” and similarly the term “polypeptide having β-N-acetylglucosaminidase activity” may be used interchangeably with the term “β-N-acetylglucosaminidase”. For purposes of the present invention, hexosaminidase activity may be determined according to the procedure described in Assay 1 or 2. In a preferred embodiment, the polypeptide having hexosaminidase activity is a dispersin. In another preferred embodiment, the polypeptide having hexosaminidase activity is a β-N-acetylglucosaminidase targeting poly-β-1,6-N-acetylglucosamine.

Expression: The term “expression” includes any step involved in the production of a variant including, but not limited to, transcription, post-transcriptional modification, translation, post-translational modification, and secretion.

Expression vector: An “expression vector” refers to a linear or circular DNA construct comprising a DNA sequence encoding a variant, which coding sequence is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences which control termination of transcription and translation.

Extension: The term “extension” means an addition of one or more amino acids to the amino and/or carboxyl terminus of a variant, wherein the “extended” variant has hexosaminidase activity.

Fragment: The term “fragment” means a variant having one or more amino acids absent from the amino and/or carboxyl terminus of the variant; wherein the fragment has hexosaminidase activity.

Host strain or host cell: A “host strain” or “host cell” is an organism into which an expression vector, phage, virus, or other DNA construct, including a polynucleotide encoding a variant has been introduced. Exemplary host strains are microorganism cells (e.g., bacteria, filamentous fungi, and yeast) capable of expressing the polypeptide of interest.

Improved property: The term “improved property” means a characteristic associated with a variant that is improved compared to the parent. Such improved properties may include, but are not limited to, catalytic efficiency, catalytic rate, chemical stability, oxidation stability, pH activity, pH stability, specific activity, stability under storage conditions, substrate binding, substrate cleavage, substrate specificity, substrate stability, surface properties, thermal activity, and thermostability. In the context of the present invention, the improved property is preferably improved stability, e.g. improved thermostability or improved storage stability in a detergent composition. The improved property, e.g. improved stability, of a hexosaminidase variant of the present invention may be determined as described in the examples herein.

Isolated: The term “isolated” means a variant, nucleic acid, cell, or other specified material or component that is separated from at least one other material or component, including but not limited to, other proteins, nucleic acids, cells, etc. An isolated polypeptide, nucleic acid, cell or other material is thus in a form that does not occur in nature. An isolated polypeptide includes, but is not limited to, a culture broth containing the secreted variant expressed in a host cell.

Mature polypeptide: The term “mature polypeptide” means a polypeptide in its mature form following N-terminal processing and/or C-terminal processing (e.g., removal of signal peptide).

Mature polypeptide coding sequence: The term “mature polypeptide coding sequence” means a polynucleotide that encodes a mature polypeptide having hexosaminidase activity.

Mutant: The term “mutant” means a polynucleotide encoding a variant.

Native: The term “native” means a nucleic acid or polypeptide naturally occurring in a host cell.

Nucleic acid: The term “nucleic acid” encompasses DNA, RNA, heteroduplexes, and synthetic molecules capable of encoding a variant. Nucleic acids may be single stranded or double stranded, and may be chemical modifications. The terms “nucleic acid” and “polynucleotide” are used interchangeably. Because the genetic code is degenerate, more than one codon may be used to encode a particular amino acid, and the present compositions and methods encompass nucleotide sequences that encode a particular amino acid sequence. Unless otherwise indicated, nucleic acid sequences are presented in 5-to-3′ orientation.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, and which comprises one or more control sequences operably linked to the nucleic acid sequence.

Operably linked: The term “operably linked” means that specified components are in a relationship (including but not limited to juxtaposition) permitting them to function in an intended manner. For example, a regulatory sequence is operably linked to a coding sequence such that expression of the coding sequence is under control of the regulatory sequence.

Parent or parent hexosaminidase: The term “parent”, “parent hexosaminidase” or “parent enzyme” means a hexosaminidase to which an alteration is made to produce the enzyme variants of the present invention. Thus, the parent is a hexosaminidase polypeptide having the identical amino acid sequence of a variant disclosed herein but not having the specified alterations, typically substitutions, disclosed herein.

In a particular embodiment the hexosaminidase parent is a hexosaminidase with at least 60%, at least 61%, at least 62%, at least 63%, at least 64%, at least 65%, at least 66%, at least 67%, at least 68%, at least 69%, at least 70%, at least 72%, at least 73%, at least 74%, at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99%, at least 99.5% or 100% identity to SEQ ID NO: 1, SEQ ID NO: 2 or SEQ ID NO: 3.

In one embodiment, the parent hexosaminidase is a hexosaminidase having the sequence of SEQ ID NO: 1, SEQ ID NO: 2 or SEQ ID NO: 3. In a preferred embodiment, the parent hexosaminidase is the polypeptide of SEQ ID NO: 1.

Recombinant: The term “recombinant” is used in its conventional meaning to refer to the manipulation, e.g., cutting and rejoining, of nucleic acid sequences to form constellations different from those found in nature. The term recombinant refers to a cell, nucleic acid, variant or vector that has been modified from its native state. Thus, for example, recombinant cells express genes that are not found within the native (non-recombinant) form of the cell, or express native genes at different levels or under different conditions than found in nature. The term “recombinant” is synonymous with “genetically modified” and “transgenic”.

Sequence identity: The relatedness between two amino acid sequences or between two nucleotide sequences is described by the parameter “sequence identity”.

The sequence identity between two amino acid sequences is determined as the output of “longest identity” using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), version 6.6.0. The parameters used are a gap open penalty of 10, a gap extension penalty of 0.5, and the EBLOSUM62 (EMBOSS version of BLOSUM62) substitution matrix. In order for the Needle program to report the longest identity, the -nobrief option must be specified in the command line. The output of Needle labeled “longest identity” is calculated as follows:

(Identical Residues×100)/(Length of Alignment−Total Number of Gaps in Alignment)

The sequence identity between two polynucleotide sequences is determined as the output of “longest identity” using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), version 6.6.0. The parameters used are a gap open penalty of 10, a gap extension penalty of 0.5, and the EDNAFULL (EMBOSS version of NCBI NUC4.4) substitution matrix. In order for the Needle program to report the longest identity, the nobrief option must be specified in the command line. The output of Needle labeled “longest identity” is calculated as follows:

(Identical Deoxyribonucleotides×100)/(Length of Alignment−Total Number of Gaps in Alignment)

Variant: The term “variant” means a polypeptide having hexosaminidase activity comprising a substitution, an insertion (including extension), and/or a deletion (e.g., truncation), at one or more positions. A substitution means replacement of the amino acid occupying a position with a different amino acid; a deletion means removal of the amino acid occupying a position; and an insertion means adding 1-5 amino acids (e.g., 1-3 amino acids, in particular, 1 amino acid) adjacent to and immediately following the amino acid occupying a position.

Wild-type: The term “wild-type” in reference to an amino acid sequence or nucleic acid sequence means that the amino acid sequence or nucleic acid sequence is a native or naturally occurring sequence. As used herein, the term “naturally occurring” refers to anything (e.g., proteins, amino acids, or nucleic acid sequences) that is found in nature. Conversely, the term “non-naturally occurring” refers to anything that is not found in nature (e.g., recombinant nucleic acids and protein sequences produced in the laboratory or modification of the wild-type sequence).

Biofilm: A biofilm is any group of microorganisms in which cells stick to each other on a surface, such as a textile or a dishware or other hard surface. These adherent cells are frequently embedded within a self-produced matrix of extracellular polymeric substance (EPS). Biofilm EPS is a polymeric conglomeration generally composed of extracellular DNA, proteins and polysaccharides. Biofilms may form on living or non-living surfaces. The microbial cells growing in a biofilm are physiologically distinct from planktonic cells of the same organism, which, by contrast, are single cells that may float or swim in a liquid medium. Bacteria living in a biofilm usually have significantly different properties from free-floating bacteria of the same species, as the dense and protected environment of the film allows them to cooperate and interact in various ways. One benefit of this environment is increased resistance to detergents and antibiotics, as the dense extracellular matrix and the outer layer of cells protect the interior of the community. On laundry, biofilm producing bacteria can e.g. be found among the following species: Acinetobacter sp., Aeromicrobium sp., Brevundimonas sp., Microbacterium sp., Micrococcus luteus, Pseudomonas sp., Staphylococcus epidermidis, and Stenotrophomonas sp.

Stability: The term “stability” includes storage stability and stability during use, e.g. during a wash process, and reflects the stability of the hexosaminidase variant according to the invention as a function of time, e.g. how much activity is retained when the hexosaminidase variant is stored either in dry form or in solution, for example in a detergent solution. The stability is influenced by many factors such as pH, temperature, and the nature of the detergent composition, e.g. amount and type of builder, surfactants etc. The hexosaminidase stability may be measured as described in the examples and expressed e.g. as a half-life improvement factor (HIF) or a melting temperature (Tm) compared to the parent hexosaminidase or a reference hexosaminidase such as the polypeptide of SEQ ID NO: 1, SEQ ID NO: 2 or SEQ ID NO: 3. The term “improved stability” or “increased stability” is defined herein as a variant hexosaminidase displaying an increased stability in solutions, relative to the stability of the parent hexosaminidase without the substitutions in the variant and/or relative to SEQ ID NO: 1, SEQ ID NO: 2 and SEQ ID NO: 3. The hexosaminidases having SEQ ID NO: 2 and SEQ ID NO: 3 have improved stability (Tm and HIF) over the hexosaminidase of SEQ ID NO: 1; thus, hexosaminidase variants with improved stability compared to SEQ ID NO: 2 or 3 will also have improved stability over SEQ ID NO: 1. The terms “improved stability” and “increased stability” include detergent stability.

Wash performance: The term “wash performance” in the context of the invention refers to a cleaning effect in laundry, preferably a deep cleaning effect, where “deep cleaning” refers to the disruption or removal of a biofilm or components thereof, of a hexosaminidase variant of the invention compared to the hexosaminidase parent or the hexosaminidase with SEQ ID NO: 1.

Wash performance may be expressed as a remission value of stained swatches. After washing and rinsing the swatches are spread out flat and allowed to air dry at room temperature overnight. All washed swatches are evaluated the day after washing. Light reflectance evaluations of the swatches may be performed using a Macbeth Color Eye 7000 reflectance spectrophotometer with very small aperture. The measurements are made without UV in the incident light and remission value at 460 nm is extracted.

Laundering: The term “laundering” relates to both household laundering and industrial laundering and means the process of treating textiles with a solution containing e.g. a cleaning or detergent composition of the present invention. The laundering process can for example be carried out using a household or an industrial washing machine or can be carried out by hand.

Detergent composition: The term “detergent composition” (or “cleaning composition”) includes unless otherwise indicated any form of detergent or cleaning composition. These include granular or powder-form all-purpose or heavy-duty washing agents, especially cleaning detergents; liquid, gel or paste-form all-purpose washing agents, especially the so-called heavy-duty liquid (HDL) types; single unit dose (SUD) compositions such as pods, capsules, tabs, etc. with one or more chambers; liquid fine-fabric detergents; hand dishwashing agents or light duty dishwashing agents, especially those of the high-foaming type; machine dishwashing agents, including the various tablet, granular, liquid and rinse-aid types for household and institutional use; liquid cleaning and disinfecting agents, including antibacterial hand-wash types, cleaning bars, soap bars, mouthwashes, denture cleaners, car or carpet shampoos, bathroom cleaners; hair shampoos and hair-rinses; shower gels, foam baths; metal cleaners; as well as cleaning auxiliaries such as bleach additives and “stain-stick” or pre-treat types. The terms “detergent composition” and “detergent formulation” are used in reference to mixtures which are intended for use in a wash medium for the cleaning of soiled objects. In some embodiments, the term is used in reference to laundering fabrics and/or garments (e.g., “laundry detergents”). In alternative embodiments, the term refers to other detergents, such as those used to clean dishes, cutlery, etc. (e.g., “dishwashing detergents”). It is not intended that the present invention be limited to any particular detergent formulation or composition. The term “detergent composition” is not intended to be limited to compositions that contain surfactants. It is intended that in addition to the variants according to the invention, the term encompasses detergents that may contain, e.g., surfactants, builders, chelators or chelating agents, bleach system or bleach components, polymers, fabric conditioners, foam boosters, suds suppressors, dyes, perfume, tannish inhibitors, optical brighteners, bactericides, fungicides, soil suspending agents, anti-corrosion agents, enzyme inhibitors or stabilizers, enzyme activators, transferases, hydrolytic enzymes, oxido reductases, bluing agents and fluorescent dyes, antioxidants, and solubilizers.

Fabric: The term “fabric” encompasses any textile material. Thus, it is intended that the term encompass garments, as well as fabrics, yarns, fibers, non-woven materials, natural materials, synthetic materials, and any other textile material.

Textile: The term “textile” refers to woven fabrics, as well as staple fibers and filaments suitable for conversion to or use as yarns, woven, knit, and non-woven fabrics. The term encompasses yarns made from natural, as well as synthetic (e.g., manufactured) fibers. The term “textile materials” is a general term for fibers, yarn intermediates, yarn, fabrics, and products made from fabrics (e.g., garments and other articles).

Non-fabric detergent compositions: The term “non-fabric detergent compositions” include non-textile surface detergent compositions, including but not limited to compositions for hard surface cleaning, such as dishwashing detergent compositions including manual dishwashing compositions, oral detergent compositions, denture detergent compositions, and personal cleansing compositions. Another group of non-fabric detergent compositions are compositions for medical cleaning, i.e. for cleaning of a medical device to remove or prevent biofilm, or for coating a medical device such as an indwelling medical device or implant to prevent biofilm formation. The medical device may be, for example, a catheter, a mechanical heart valve, a cardiac pacemaker, an arteriovenous shunt, a scleral buckle, a prosthetic joint, a tympanostomy tube, a tracheostomy tube, a voice prosthetic, a penile prosthetic, an artificial urinary sphincter, a synthetic pubovaginal sling, a surgical suture, a bone anchor, a bone screw, an intraocular lens, a contact lens, an intrauterine device, an aortofemoral graft, a vascular graft, a needle, a Luer-Lok connector, a needleless connector or a surgical instrument.

Effective amount of enzyme: The term “effective amount of enzyme” refers to the quantity of enzyme necessary to achieve the enzymatic activity required in the specific application, e.g., in a defined detergent composition. Such effective amounts are readily ascertained by one of ordinary skill in the art and are based on many factors, such as the particular enzyme used, the cleaning application, the specific composition of the detergent composition, and whether a liquid or dry (e.g., granular, bar) composition is required, and the like. The term “effective amount” of a hexosaminidase variant refers to the quantity of hexosaminidase variant described hereinbefore that achieves a desired level of enzymatic activity, e.g., in a defined detergent composition.

Relevant washing conditions: The term “relevant washing conditions” is used herein to indicate the conditions, particularly washing temperature, time, washing mechanics, detergent concentration, type of detergent and water hardness, actually used in households in a detergent market segment.

Wash liquor: The term “wash liquor” (or “wash water”) refers to an aqueous solution comprising a hexosaminidase variant of the invention. A wash liquor is a solution, e.g. found in a washing machine or dishwasher, containing water and a detergent composition comprising the hexosaminidase variant. The detergent composition, prior to being mixed with water to form a wash liquor, may be in any suitable form as described elsewhere herein, for example a liquid or powder.

Water hardness: The term “water hardness” or “degree of hardness” or “dH” or “° dH” as used herein refers to German degrees of hardness. One degree is defined as 10 milligrams of calcium oxide per liter of water.

Adjunct materials: The term “adjunct materials” or “adjunct ingredients” means any liquid, solid or gaseous material selected for the particular type of detergent composition desired and the form of the product (e.g., liquid, granule, powder, bar, paste, spray, tablet, gel, or foam composition), which materials are also preferably compatible with the hexosaminidase variant enzyme used in the composition. More detailed information on adjunct materials is provided further below.

Low detergent concentration: The term “low detergent concentration” system includes detergents where less than about 800 ppm of detergent components is present in the wash water. Asian, e.g., Japanese detergents are typically considered low detergent concentration systems.

Medium detergent concentration: The term “medium detergent concentration” system includes detergents wherein between about 800 ppm and about 2000 ppm of detergent components is present in the wash water. North American detergents are generally considered to be medium detergent concentration systems.

High detergent concentration: The term “high detergent concentration” system includes detergents wherein greater than about 2000 ppm of detergent components is present in the wash water. European detergents are generally considered to be high detergent concentration systems.

### Conventions for Designation of Variants

For purposes of the present invention, the polypeptide disclosed in SEQ ID NO: 1 is used to determine the corresponding amino acid positions in another hexosaminidase. The amino acid sequence of another hexosaminidase is aligned with the polypeptide disclosed in SEQ ID NO: 1, and based on the alignment, the amino acid position number corresponding to any amino acid residue in the polypeptide disclosed in SEQ ID NO: 1 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix.

In describing the variants of the present invention, the nomenclature described below is adapted for ease of reference. The accepted IUPAC single letter or three letter amino acid abbreviation is employed.

Substitutions. For an amino acid substitution, the following nomenclature is used: Original amino acid, position, substituted amino acid. Accordingly, the substitution of threonine at position 226 with alanine is designated as “T226A” (or “Thr226Ala” using the three-letter code). Multiple mutations may be separated by addition marks (“+”), e.g., “G205R+S411F”, representing substitutions at positions 205 and 411 of glycine (G) with arginine (R) and serine (S) with phenylalanine (F), respectively. Multiple mutations may alternatively be indicated by a space, a comma, or a plus sign, e.g. “G205R S411F”, “G205R, S411F” or “G205R+S411F”.

Deletions. For an amino acid deletion, the following nomenclature is used: Original amino acid, position, *. Accordingly, the deletion of glycine at position 195 is designated as “G195*”. Multiple deletions are separated by addition marks (“+”), e.g., “G195*+S411*”.

Insertions. For an amino acid insertion, the following nomenclature is used: Original amino acid, position, original amino acid, inserted amino acid. Accordingly, the insertion of lysine after glycine at position 195 is designated “G195GK”. An insertion of multiple amino acids is designated [Original amino acid, position, original amino acid, inserted amino acid #1, inserted amino acid #2; etc.]. For example, the insertion of lysine and alanine after glycine at position 195 is indicated as “G195GKA”.

In such cases the inserted amino acid residue(s) are numbered by the addition of lower-case letters to the position number of the amino acid residue preceding the inserted amino acid residue(s). In the above example, the sequence would thus be:

| Parent: | Variant: |
|---|---|
| 195 | 195 195a 195b |
| G | G - K - A |

Multiple alterations. Variants comprising multiple alterations are separated by addition marks (“+”) as explained above, e.g., “R170Y+G195E” representing a substitution of arginine and glycine at positions 170 and 195 with tyrosine and glutamic acid, respectively. Alternatively, the multiple alterations may be separated by a space or comma as mentioned above.

Different alterations. Where different alterations can be introduced at a position, the different alterations may be separated by a comma or a slash, e.g., “R170Y,E” or “R170Y/E” represents a substitution of arginine at position 170 with tyrosine or glutamic acid. Thus, “Y167G,A+R170G,A” or “Y167G/A+R170G/A” designates the following variants: “Y167G+R170G”, “Y167G+R170A”, “Y167A+R170G” and “Y167A+R170A”.

### DETAILED DESCRIPTION OF THE INVENTION

The present invention provides variants of the hexosaminidase polypeptide of SEQ ID NO: 1, wherein the variant comprises compared to SEQ ID NO: 1:
a substitution at one or more positions, preferably two, three or four positions, corresponding to positions 163, 227, 252 and 309 of SEQ ID NO: 1; and
a substitution at one or more positions corresponding to positions 106, 111, 120, 124, 127, 150, 170, 171, 178, 199, 208, 254, 255 and 278 of SEQ ID NO: 1;
wherein the variant has hexosaminidase activity and has at least 60%, e.g., at least 65%, at least 70%, at least 75%, at least 80%, at least 85%, at least 90% or at least 95% sequence identity, but less than 100% sequence identity, to the polypeptide of SEQ ID NO: 1, SEQ ID NO: 2 or SEQ ID NO: 3.

### Variants

In an embodiment, the variant of the invention comprises, compared to SEQ ID NO: 1:
one or more, e.g. two, three or four, of the indicated amino acid residues in the following positions: P in position 163, T in position 227, P in position 252 and E in position 309; and
one or more of the indicated amino acid residues in the following positions: M or N in position 106, R in position 111, V in position 120, 1, K or L in position 124, P in position 127, N in position 150, G in position 170, F or H in position 171, K in position 178, W in position 199, W in position 208, I in position 254, D in position 255 and V in position 278;
wherein position numbers are based on SEQ ID NO: 1.

The hexosaminidase variant may thus comprise, compared to SEQ ID NO: 1, two, three or four substitutions selected from the group consisting of S163P, N227T, N252P and K309E;
and at least one substitution selected from the group consisting of V106M, V106N, D111R, T120V, Y124I, Y124K, Y124L, R127P, E150N, L170G, D171F, D171H, Q178K, S199W, S208W, F254I, T255D and I278V.

In preferred embodiments, the hexosaminidase variant may further comprise, compared to SEQ ID NO: 1, at least one substitution selected from the group consisting of H15Y, S186R, S225G, E232D, G235W, N260Q, H272V, S279D, Y281P, K308Q and K312Q, for example 2, 3, 4, 5, 6, 7, 8, 9, 10 or all of said substitutions. In such embodiments, the variant may in addition comprise, compared to SEQ ID NO: 1, one or more substitutions selected from the group consisting of Q3I, A49W and N59E, preferably 2 or all 3 of said substitutions; or one or more substitutions selected from the group consisting of Q3F, V140I, Q215K and N267T, preferably 2, 3 or all 4 of said substitutions.

In one embodiment, the hexosaminidase variant comprises, compared to SEQ ID NO: 1: the substitutions S163P, N227T, N252P and K309E; and at least one substitution selected from the group consisting of H15Y, S186R, S225G, E232D, G235W, N260Q, H272V, S279D, Y281P, K308Q and K312Q, for example 2, 3, 4, 5, 6, 7, 8, 9, 10 or all of said substitutions.

In preferred embodiments, the hexosaminidase variant of the invention comprises, compared to SEQ ID NO: 1, at least one substitution selected from the group consisting of V106M/N, D111R, T120V, Y124I/K/L, R127P, E150N, L170G, D171F/H, Q178K, S199W, S208W, T255D, N267T, I278V and K308E. Preferably, the variant comprises at least two of said substitutions, e.g. at least three of said substitutions. One such variant is a variant of SEQ ID NO: 2 comprising at least one substitution selected from the group consisting of V106M/N, D111R, T120V, Y124I/K/L, R127P, E150N, L170G, D171F/H, Q178K, S199W, S208W, T255D, N267T, I278V and K308E, for example at least two or at least three of said substitutions. Another such variant is a variant of SEQ ID NO: 3 comprising at least one substitution selected from the group consisting of V106M/N, D111R, T120V, Y124I/K/L, R127P, E150N, L170G, D171F/H, Q178K, S199W, S208W, T255D, N267T, I278V and K308E, for example at least two or at least three of said substitutions.

In more preferred embodiments, the hexosaminidase variant of the invention comprises, compared to SEQ ID NO: 1, at least one substitution selected from the group consisting of V106M/N, D111R, T120V, E150N, L170G, D171F, Q178K, S199W, S208W, T255D and I278V. Preferably, the variant comprises at least two of said substitutions, e.g. at least three of said substitutions. One such variant is a variant of SEQ ID NO: 2 comprising at least one substitution selected from the group consisting of V106M/N, D111R, T120V, E150N, L170G, D171F, Q178K, S199W, S208W, T255D and I278V, for example at least two or at least three of said substitutions. Another such variant is a variant of SEQ ID NO: 3 comprising at least one substitution selected from the group consisting of V106M/N, D111R, T120V, E150N, L170G, D171F, Q178K, S199W, S208W, T255D and I278V, for example at least two or at least three of said substitutions.

More preferably, the hexosaminidase variant of the invention comprises, compared to SEQ ID NO: 1, at least one substitution selected from the group consisting of V106M/N, D111R, T120V, E150N, D171F and I278V, for example at least two of said substitutions, e.g. at least three of said substitutions.

In particularly preferred embodiments, the hexosaminidase variant of the invention comprises, compared to SEQ ID NO: 1, at least one substitution selected from the group consisting of D111R, T120V and E150N. The variant may thus comprise the substitutions D111R+T120V, D111R+E150N or T120V+E150N, or all three substitutions D111R+T120V+E150N. In these embodiments, the variant may in addition comprise at least one substitution selected from the group consisting of V106M, V106N, Y124I, Y124K, Y124L, R127P, L170G, D171F, D171H, Q178K, S199W, S208W, F254I, T255D and I278V, where preferred substitutions include those selected from the group consisting of V106M/N, L170G, D171F, Q178K, S199W, S208W, T255D and I278V.

In one embodiment, the hexosaminidase variant comprises, compared to SEQ ID NO: 1, the substitution D111R together with at least one substitution selected from T120V and E150N, and optionally at least one additional substitution selected from the group consisting of V106M, V106N, Y124I, Y124K, Y124L, R127P, L170G, D171F, D171H, Q178K, S199W, S208W, F254I, T255D and I278V.

In one embodiment, the hexosaminidase variant comprises, compared to SEQ ID NO: 1, the substitution T120V together with at least one substitution selected from D111R and E150N, and optionally at least one additional substitution selected from the group consisting of V106M, V106N, Y124I, Y124K, Y124L, R127P, L170G, D171F, D171H, Q178K, S199W, S208W, F254I, T255D and I278V.

In one embodiment, the hexosaminidase variant comprises, compared to SEQ ID NO: 1, the substitution E150N together with at least one substitution selected from D111R and T120V, and optionally at least one additional substitution selected from the group consisting of V106M, V106N, Y124I, Y124K, Y124L, R127P, L170G, D171F, D171H, Q178K, S199W, S208W, F254I, T255D and I278V.

In some embodiments, the hexosaminidase variant comprises, compared to SEQ ID NO: 1, at least one substitution selected from the group consisting of V106M, V106N, Y124I, Y124K, Y124L, R127P, L170G, D171F, D171H, Q178K, S199W, S208W, F254I, T255D and I278V; for example selected from the group consisting of V106M/N, L170G, D171F, Q178K, S199W, S208W, T255D and I278V.

In some embodiments, the hexosaminidase variant comprises, compared to SEQ ID NO: 1, a) two, three or, preferably, four substitutions selected from the group consisting of S163P, N227T, N252P and K309E; b) at least one substitution, preferably at least 2, 3, 4, 5, 6, 7, 8, 9 or 10 substitutions, selected from the group consisting of H15Y, S186R, S225G, E232D, G235W, N260Q, H272V, S279D, Y281P, K308Q and K312Q, more preferably all of said substitutions; and c) at least one substitution selected from the group consisting of V106M, V106N, D111R, T120V, Y124I, Y124K, Y124L, R127P, E150N, 1170G, D171F, D171H, Q178K, S199W, S208W, F254I, T255D and I278V.

In one embodiment, the hexosaminidase variant comprises, compared to SEQ ID NO: 1, a) two, three or, preferably, four substitutions selected from the group consisting of S163P, N227T, N252P and K309E; b) at least one substitution, preferably at least 2, 3, 4, 5, 6, 7, 8, 9 or 10 substitutions, selected from the group consisting of H15Y, S186R, S225G, E232D, G235W, N260Q, H272V, S279D, Y281P, K308Q and K312Q, more preferably all of said substitutions; and c) the substitution V106M. Such a variant may further comprise one or more additional substitutions, e.g. selected from the group consisting of D111R, T120V, E150N, D171F and I278V.

In one embodiment, the hexosaminidase variant comprises, compared to SEQ ID NO: 1, a) two, three or, preferably, four substitutions selected from the group consisting of S163P, N227T, N252P and K309E; b) at least one substitution, preferably at least 2, 3, 4, 5, 6, 7, 8, 9 or 10 substitutions, selected from the group consisting of H15Y, S186R, S225G, E232D, G235W, N260Q, H272V, S279D, Y281P, K308Q and K312Q, more preferably all of said substitutions; and c) the substitution V106N. Such a variant may further comprise one or more additional substitutions, e.g. selected from the group consisting of D111R, T120V, E150N, D171F and I278V.

In one embodiment, the hexosaminidase variant comprises, compared to SEQ ID NO: 1, a) two, three or, preferably, four substitutions selected from the group consisting of S163P, N227T, N252P and K309E; b) at least one substitution, preferably at least 2, 3, 4, 5, 6, 7, 8, 9 or 10 substitutions, selected from the group consisting of H15Y, S186R, S225G, E232D, G235W, N260Q, H272V, S279D, Y281P, K308Q and K312Q, more preferably all of said substitutions; and c) the substitution D111R. Such a variant may further comprise one or more additional substitutions, e.g. selected from the group consisting of V106M/N, T120V, E150N, D171F and I278V.

In one embodiment, the hexosaminidase variant comprises, compared to SEQ ID NO: 1, a) two, three or, preferably, four substitutions selected from the group consisting of S163P, N227T, N252P and K309E; b) at least one substitution, preferably at least 2, 3, 4, 5, 6, 7, 8, 9 or 10 substitutions, selected from the group consisting of H15Y, S186R, S225G, E232D, G235W, N260Q, H272V, S279D, Y281P, K308Q and K312Q, more preferably all of said substitutions; and c) the substitution T120V. Such a variant may further comprise one or more additional substitutions, e.g. selected from the group consisting of V106M/N, D111R, E150N, D171F and I278V.

In one embodiment, the hexosaminidase variant comprises, compared to SEQ ID NO: 1, a) two, three or, preferably, four substitutions selected from the group consisting of S163P, N227T, N252P and K309E; b) at least one substitution, preferably at least 2, 3, 4, 5, 6, 7, 8, 9 or 10 substitutions, selected from the group consisting of H15Y, S186R, S225G, E232D, G235W, N260Q, H272V, S279D, Y281P, K308Q and K312Q, more preferably all of said substitutions; and c) the substitution Y124I. Such a variant may further comprise one or more additional substitutions, e.g. selected from the group consisting of V106M/N, D111R, T120V, E150N, D171F and I278V.

In one embodiment, the hexosaminidase variant comprises, compared to SEQ ID NO: 1, a) two, three or, preferably, four substitutions selected from the group consisting of S163P, N227T, N252P and K309E; b) at least one substitution, preferably at least 2, 3, 4, 5, 6, 7, 8, 9 or 10 substitutions, selected from the group consisting of H15Y, S186R, S225G, E232D, G235W, N260Q, H272V, S279D, Y281P, K308Q and K312Q, more preferably all of said substitutions; and c) the substitution Y124K. Such a variant may further comprise one or more additional substitutions, e.g. selected from the group consisting of V106M/N, D111R, T120V, E150N, D171F and I278V.

In one embodiment, the hexosaminidase variant comprises, compared to SEQ ID NO: 1, a) two, three or, preferably, four substitutions selected from the group consisting of S163P, N227T, N252P and K309E; b) at least one substitution, preferably at least 2, 3, 4, 5, 6, 7, 8, 9 or 10 substitutions, selected from the group consisting of H15Y, S186R, S225G, E232D, G235W, N260Q, H272V, S279D, Y281P, K308Q and K312Q, more preferably all of said substitutions; and c) the substitution Y124L. Such a variant may further comprise one or more additional substitutions, e.g. selected from the group consisting of V106M/N, D111R, T120V, E150N, D171F and I278V.

In one embodiment, the hexosaminidase variant comprises, compared to SEQ ID NO: 1, a) two, three or, preferably, four substitutions selected from the group consisting of S163P, N227T, N252P and K309E; b) at least one substitution, preferably at least 2, 3, 4, 5, 6, 7, 8, 9 or 10 substitutions, selected from the group consisting of H15Y, S186R, S225G, E232D, G235W, N260Q, H272V, S279D, Y281P, K308Q and K312Q, more preferably all of said substitutions; and c) the substitution R127P. Such a variant may further comprise one or more additional substitutions, e.g. selected from the group consisting of V106M/N, D111R, T120V, E150N, D171F and I278V.

In one embodiment, the hexosaminidase variant comprises, compared to SEQ ID NO: 1, a) two, three or, preferably, four substitutions selected from the group consisting of S163P, N227T, N252P and K309E; b) at least one substitution, preferably at least 2, 3, 4, 5, 6, 7, 8, 9 or 10 substitutions, selected from the group consisting of H15Y, S186R, S225G, E232D, G235W, N260Q, H272V, S279D, Y281P, K308Q and K312Q, more preferably all of said substitutions; and c) the substitution E150N. Such a variant may further comprise one or more additional substitutions, e.g. selected from the group consisting of V106M/N, D111R, T120V, E150N, D171F and I278V. Such a variant may further comprise one or more additional substitutions, e.g. selected from the group consisting of V106M/N, D111R, T120V, D171F and I278V.

In one embodiment, the hexosaminidase variant comprises, compared to SEQ ID NO: 1, a) two, three or, preferably, four substitutions selected from the group consisting of S163P, N227T, N252P and K309E; b) at least one substitution, preferably at least 2, 3, 4, 5, 6, 7, 8, 9 or 10 substitutions, selected from the group consisting of H15Y, S186R, S225G, E232D, G235W, N260Q, H272V, S279D, Y281P, K308Q and K312Q, more preferably all of said substitutions; and c) the substitution L170G. Such a variant may further comprise one or more additional substitutions, e.g. selected from the group consisting of V106M/N, D111R, T120V, E150N, D171F and I278V. Such a variant may further comprise one or more additional substitutions, e.g. selected from the group consisting of V106M/N, D111R, T120V, E150N, D171F and I278V.

In one embodiment, the hexosaminidase variant comprises, compared to SEQ ID NO: 1, a) two, three or, preferably, four substitutions selected from the group consisting of S163P, N227T, N252P and K309E; b) at least one substitution, preferably at least 2, 3, 4, 5, 6, 7, 8, 9 or 10 substitutions, selected from the group consisting of H15Y, S186R, S225G, E232D, G235W, N260Q, H272V, S279D, Y281P, K308Q and K312Q, more preferably all of said substitutions; and c) the substitution D171F. Such a variant may further comprise one or more additional substitutions, e.g. selected from the group consisting of V106M/N, D111R, T120V, E150N and I278V.

In one embodiment, the hexosaminidase variant comprises, compared to SEQ ID NO: 1, a) two, three or, preferably, four substitutions selected from the group consisting of S163P, N227T, N252P and K309E; b) at least one substitution, preferably at least 2, 3, 4, 5, 6, 7, 8, 9 or 10 substitutions, selected from the group consisting of H15Y, S186R, S225G, E232D, G235W, N260Q, H272V, S279D, Y281P, K308Q and K312Q, more preferably all of said substitutions; and c) the substitution D171H. Such a variant may further comprise one or more additional substitutions, e.g. selected from the group consisting of V106M/N, D111R, T120V, E150N and I278V.

In one embodiment, the hexosaminidase variant comprises, compared to SEQ ID NO: 1, a) two, three or, preferably, four substitutions selected from the group consisting of S163P, N227T, N252P and K309E; b) at least one substitution, preferably at least 2, 3, 4, 5, 6, 7, 8, 9 or 10 substitutions, selected from the group consisting of H15Y, S186R, S225G, E232D, G235W, N260Q, H272V, S279D, Y281P, K308Q and K312Q, more preferably all of said substitutions; and c) the substitution Q178K. Such a variant may further comprise one or more additional substitutions, e.g. selected from the group consisting of V106M/N, D111R, T120V, E150N, D171F and I278V.

In one embodiment, the hexosaminidase variant comprises, compared to SEQ ID NO: 1, a) two, three or, preferably, four substitutions selected from the group consisting of S163P, N227T, N252P and K309E; b) at least one substitution, preferably at least 2, 3, 4, 5, 6, 7, 8, 9 or 10 substitutions, selected from the group consisting of H15Y, S186R, S225G, E232D, G235W, N260Q, H272V, S279D, Y281P, K308Q and K312Q, more preferably all of said substitutions; and c) the substitution S199W. Such a variant may further comprise one or more additional substitutions, e.g. selected from the group consisting of V106M/N, D111R, T120V, E150N, D171F and I278V.

In one embodiment, the hexosaminidase variant comprises, compared to SEQ ID NO: 1, a) two, three or, preferably, four substitutions selected from the group consisting of S163P, N227T, N252P and K309E; b) at least one substitution, preferably at least 2, 3, 4, 5, 6, 7, 8, 9 or 10 substitutions, selected from the group consisting of H15Y, S186R, S225G, E232D, G235W, N260Q, H272V, S279D, Y281P, K308Q and K312Q, more preferably all of said substitutions; and c) the substitution S208W. Such a variant may further comprise one or more additional substitutions, e.g. selected from the group consisting of V106M/N, D111R, T120V, E150N, D171F and I278V.

In one embodiment, the hexosaminidase variant comprises, compared to SEQ ID NO: 1, a) two, three or, preferably, four substitutions selected from the group consisting of S163P, N227T, N252P and K309E; b) at least one substitution, preferably at least 2, 3, 4, 5, 6, 7, 8, 9 or 10 substitutions, selected from the group consisting of H15Y, S186R, S225G, E232D, G235W, N260Q, H272V, S279D, Y281P, K308Q and K312Q, more preferably all of said substitutions; and c) the substitution F254I. Such a variant may further comprise one or more additional substitutions, e.g. selected from the group consisting of V106M/N, D111R, T120V, E150N, D171F and I278V.

In one embodiment, the hexosaminidase variant comprises, compared to SEQ ID NO: 1, a) two, three or, preferably, four substitutions selected from the group consisting of S163P, N227T, N252P and K309E; b) at least one substitution, preferably at least 2, 3, 4, 5, 6, 7, 8, 9 or 10 substitutions, selected from the group consisting of H15Y, S186R, S225G, E232D, G235W, N260Q, H272V, S279D, Y281P, K308Q and K312Q, more preferably all of said substitutions; and c) the substitution T255D. Such a variant may further comprise one or more additional substitutions, e.g. selected from the group consisting of V106M/N, D111R, T120V, E150N, D171F and I278V.

In one embodiment, the hexosaminidase variant comprises, compared to SEQ ID NO: 1, a) two, three or, preferably, four substitutions selected from the group consisting of S163P, N227T, N252P and K309E; b) at least one substitution, preferably at least 2, 3, 4, 5, 6, 7, 8, 9 or 10 substitutions, selected from the group consisting of H15Y, S186R, S225G, E232D, G235W, N260Q, H272V, S279D, Y281P, K308Q and K312Q, more preferably all of said substitutions; and c) the substitution I278V. Such a variant may further comprise one or more additional substitutions, e.g. selected from the group consisting of V106M/N, D111R, T120V, E150N and D171F.

Examples of specific hexosaminidase variants of the invention include variants which compared to SEQ ID NO: 1, SEQ ID NO: 2 or SEQ ID NO: 3, preferably compared to SEQ ID NO: 2, comprise at least one of the following substitutions or sets of substitutions:
D111R
D111R+T120V
D111R+T120V+E150N
D111R+T120V+E150N+D171F+I278V
D111R+T120V+T255D
D111R+T120V+Y124I
D111R+T120V+Y124K+Q178K
D111R+T120V+Y124L+Q178K
D111R+Y124K+D171F
D171F+S208W+N267T
D171F+T255D
D171H
I278V
K308E
N267T
R127P
T120V+D171F
T120V+D171F+N267T
T120V+S208W+N267T
T120V+T255D
T120V+T255D+N267T
T120V+Y124K+Q178K
V106M+D111R+T120V+D171F
V106M+Q178K+T255D
V106M+T120V+D171F+Q178K
V106M+T120V+Y124I+D171F
V106M+T120V+Y124I+Q178K
V106M+T120V+Y124K+D171F
V106M+T120V+Y124L+Q178K
V106N+D111R+T120V+E150N
V106N+S208W+N267T
V106N+T120V+D171F
V106N+T120V+D171F+S208W
V106N+T120V+E150N+D171F+I278V
V106N+T120V+N267T

The specific hexosaminidase variants listed above may have a sequence identity compared to SEQ ID NO: 2 or SEQ ID NO: 3, preferably compared to SEQ ID NO: 2, of at least 80%, preferably at least 85%, more preferably at least 90%, such as at least 91%, at least 92%, at least 93%, at least 94%, at least 95%, at least 96%, at least 97%, at least 98% or at least 99%.

Other examples of specific hexosaminidase variants of the invention include variants which compared to SEQ ID NO: 1, SEQ ID NO: 2 or SEQ ID NO: 3, preferably compared to SEQ ID NO: 3, comprise at least one of the following substitutions or sets of substitutions:
L170G
E150N+F254I
F254I
V106N
D111R+T120V
D111R+T120V+E150N
D111R+T120V+E150N+D171F+I278V
D111R+T120V+T255D
D111R+T120V+Y124I
D111R+T120V+Y124K+Q178K
D111R+T120V+Y124L+Q178K
D111R+Y124K+D171F
D171F+S208W+N267T
D171F+T255D
D171H
I278V
K308E
N267T
R127P
T120V+D171F
T120V+D171F+N267T
T120V+S208W+N267T
T120V+T255D
T120V+T255D+N267T
T120V+Y124K+Q178K
V106M+D111R+T120V+D171F
V106M+Q178K+T255D
V106M+T120V+D171F+Q178K
V106M+T120V+Y124I+D171F
V106M+T120V+Y124I+Q178K
V106M+T120V+Y124K+D171F
V106M+T120V+Y124L+Q178K
V106N+D111R+T120V+E150N
V106N+S208W+N267T
V106N+T120V+D171F
V106N+T120V+D171F+S208W
V106N+T120V+E150N+D171F+I278V
V106N+T120V+N267T

The specific hexosaminidase variants listed above may have a sequence identity compared to SEQ ID NO: 2 or SEQ ID NO: 3, preferably compared to SEQ ID NO: 3, of at least 80%, preferably at least 85%, more preferably at least 90%, such as at least 91%, at least 92%, at least 93%, at least 94%, at least 95%, at least 96%, at least 97%, at least 98% or at least 99%.

Examples of preferred hexosaminidase variants of the invention include variants which comprise one of the following sets of substitutions compared to SEQ ID NO: 1:
Q3I+H15Y+A49W+N59E+D111R+T120V+S163P+S186R+S225G+N227T+E232D+G235W+N252P+N260Q+H272V+S279D+Y281P+K308Q+K309E+K312Q
Q3I+H15Y+A49W+N59E+D111R+T120V+E150N+S163P+S186R+S225G+N227T+E232D+G235W+N252P+N260Q+H272V+S279D+Y281P+K308Q+K309E+K312Q
Q3I+H15Y+A49W+N59E+D111R+T120V+E150N+S163P+D171F+S186R+S225G+N227T+E232D+G235W+N252P+N260Q+H272V+I278V+S279D+Y281P+K308Q+K309E+K312Q
Q3I+H15Y+A49W+N59E+D111R+T120V+S163P+S186R+S225G+N227T+E232D+G235W+N252P+T255D+N260Q+H272V+S279D+Y281P+K308Q+K309E+K312Q
Q3I+H15Y+A49W+N59E+D111R+T120V+Y124I+S163P+S186R+S225G+N227T+E232D+G235W+N252P+N260Q+H272V+S279D+Y281P+K308Q+K309E+K312Q
Q3I+H15Y+A49W+N59E+D111R+T120V+Y124K+S163P+Q178K+S186R+S225G+N227T+E232D+G235W+N252P+N260Q+H272V+S279D+Y281P+K308Q+K309E+K312Q
Q3I+H15Y+A49W+N59E+D111R+T120V+Y124L+S163P+Q178K+S186R+S225G+N227T+E232D+G235W+N252P+N260Q+H272V+S279D+Y281P+K308Q+K309E+K312Q
Q3I+H15Y+A49W+N59E+D111R+Y124K+S163P+D171F+S186R+S225G+N227T+E232D+G235W+N252P+N260Q+H272V+S279D+Y281P+K308Q+K309E+K312Q
Q3I+H15Y+A49W+N59E+S163P+D171F+S186R+S208W+S225G+N227T+E232D+G235W+N252P+N260Q+N267T+H272V+S279D+Y281P+K308Q+K309E+K312Q
Q3I+H15Y+A49W+N59E+S163P+D171F+S186R+S225G+N227T+E232D+G235W+N252P+T255D+N260Q+H272V+S279D+Y281P+K308Q+K309E+K312Q
Q3I+H15Y+A49W+N59E+S163P+D171H+S186R+S225G+N227T+E232D+G235W+N252P+N260Q+H272V+S279D+Y281P+K308Q+K309E+K312Q
Q3I+H15Y+A49W+N59E+S163P+S186R+S225G+N227T+E232D+G235W+N252P+N260Q+H272V+I278V+S279D+Y281P+K308Q+K309E+K312Q
Q3I+H15Y+A49W+N59E+S163P+S186R+S225G+N227T+E232D+G235W+N252P+N260Q+H272V+S279D+Y281P+K308E+K309E+K312Q
Q3I+H15Y+A49W+N59E+S163P+S186R+S225G+N227T+E232D+G235W+N252P+N260Q+N267T+H272V+S279D+Y281P+K308Q+K309E+K312Q
Q3I+H15Y+A49W+N59E+R127P+S163P+S186R+S225G+N227T+E232D+G235W+N252P+N260Q+H272V+S279D+Y281P+K308Q+K309E+K312Q
Q3I+H15Y+A49W+N59E+T120V+S163P+D171F+S186R+S225G+N227T+E232D+G235W+N252P+N260Q+H272V+S279D+Y281P+K308Q+K309E+K312Q
Q3I+H15Y+A49W+N59E+T120V+S163P+D171F+S186R+S225G+N227T+E232D+G235W+N252P+N260Q+N267T+H272V+S279D+Y281P+K308Q+K309E+K312Q
Q3I+H15Y+A49W+N59E+T120V+S163P+S186R+S208W+S225G+N227T+E232D+G235W+N252P+N260Q+N267T+H272V+S279D+Y281P+K308Q+K309E+K312Q
Q3I+H15Y+A49W+N59E+T120V+S163P+S186R+S225G+N227T+E232D+G235W+N252P+T255D+N260Q+H272V+S279D+Y281P+K308Q+K309E+K312Q
Q3I+H15Y+A49W+N59E+T120V+S163P+S186R+S225G+N227T+E232D+G235W+N252P+T255D+N260Q+N267T+H272V+S279D+Y281P+K308Q+K309E+K312Q
Q3I+H15Y+A49W+N59E+T120V+Y124K+S163P+Q178K+S186R+S225G+N227T+E232D+G235W+N252P+N260Q+H272V+S279D+Y281P+K308Q+K309E+K312Q
Q3I+H15Y+A49W+N59E+V106M+D111R+T120V+S163P+D171F+S186R+S225G+N227T+E232D+G235W+N252P+N260Q+H272V+S279D+Y281P+K308Q+K309E+K312Q
Q3I+H15Y+A49W+N59E+V106M+S163P+Q178K+S186R+S225G+N227T+E232D+G235W+N252P+T255D+N260Q+H272V+S279D+Y281P+K308Q+K309E+K312Q
Q3I+H15Y+A49W+N59E+V106M+T120V+S163P+D171F+Q178K+S186R+S225G+N227T+E232D+G235W+N252P+N260Q+H272V+S279D+Y281P+K308Q+K309E+K312Q
Q3I+H15Y+A49W+N59E+V106M+T120V+Y124I+S163P+D171F+S186R+S225G+N227T+E232D+G235W+N252P+N260Q+H272V+S279D+Y281P+K308Q+K309E+K312Q
Q3I+H15Y+A49W+N59E+V106M+T120V+Y124I+S163P+Q178K+S186R+S225G+N227T+E232D+G235W+N252P+N260Q+H272V+S279D+Y281P+K308Q+K309E+K312Q
Q3I+H15Y+A49W+N59E+V106M+T120V+Y124K+S163P+D171F+S186R+S225G+N227T+E232D+G235W+N252P+N260Q+H272V+S279D+Y281P+K308Q+K309E+K312Q
Q3I+H15Y+A49W+N59E+V106M+T120V+Y124L+S163P+Q178K+S186R+S225G+N227T+E232D+G235W+N252P+N260Q+H272V+S279D+Y281P+K308Q+K309E+K312Q
Q3I+H15Y+A49W+N59E+V106N+D111R+T120V+E150N+S163P+S186R+S225G+N227T+E232D+G235W+N252P+N260Q+H272V+S279D+Y281P+K308Q+K309E+K312Q
Q3I+H15Y+A49W+N59E+V106N+S163P+S186R+S208W+S225G+N227T+E232D+G235W+N252P+N260Q+N267T+H272V+S279D+Y281P+K308Q+K309E+K312Q
Q3I+H15Y+A49W+N59E+V106N+T120V+S163P+D171F+S186R+S225G+N227T+E232D+G235W+N252P+N260Q+H272V+S279D+Y281P+K308Q+K309E+K312Q
Q3I+H15Y+A49W+N59E+V106N+T120V+S163P+D171F+S186R+S208W+S225G+N227T+E232D+G235W+N252P+N260Q+H272V+S279D+Y281P+K308Q+K309E+K312Q
Q3I+H15Y+A49W+N59E+V106N+T120V+E150N+S163P+D171F+S186R+S225G+N227T+E232D+G235W+N252P+N260Q+H272V+I278V+S279D+Y281P+K308Q+K309E+K312Q
Q3I+H15Y+A49W+N59E+V106N+T120V+S163P+S186R+S225G+N227T+E232D+G235W+N252P+N260Q+N267T+H272V+S279D+Y281P+K308Q+K309E+K312Q
Q3F+H15Y+V140I+S163P+L170G+S186R+Q215K+S225G+N227T+E232D+G235W+N252P+N260Q+N267T+H272V+S279D+Y281P+K308Q+K309E+K312Q
Q3F+H15Y+V140I+E150N+S163P+S186R+Q215K+S225G+N227T+E232D+G235W+N252P+F254I+N260Q+N267T+H272V+S279D+Y281P+K308Q+K309E+K312Q
Q3F+H15Y+V140I+S163P+S186R+Q215K+S225G+N227T+E232D+G235W+N252P+F254I+N260Q+N267T+H272V+S279D+Y281P+K308Q+K309E+K312Q
Q3F+H15Y+V106N+V140I+S163P+S186R+Q215K+S225G+N227T+E232D+G235W+N252P+N260Q+N267T+H272V+S279D+Y281P+K308Q+K309E+K312Q
Q3F+H15Y+D111R+V140I+S163P+S186R+Q215K+S225G+N227T+E232D+G235W+N252P+N260Q+N267T+H272V+S279D+Y281P+K308Q+K309E+K312Q
Q3F+H15Y+D111R+V140I+E150N+S163P+S186R+Q215K+S225G+N227T+E232D+G235W+N252P+N260Q+N267T+H272V+S279D+Y281P+K308Q+K309E+K312Q
Q3F+H15Y+D111R+V140I+E150N+S163P+L170G+S186R+Q215K+S225G+N227T+E232D+G235W+N252P+N260Q+N267T+H272V+S279D+Y281P+K308Q+K309E+K312Q
Q3F+H15Y+D111R+V140I+E150N+S163P+L170G+S186R+Q215K+S225G+N227T+E232D+G235W+N252P+N260Q+N267T+H272V+I278V+S279D+Y281P+K308Q+K309E+K312Q
Q3F+H15Y+D111R+V140I+E150N+S163P+S186R+S199W+Q215K+S225G+N227T+E232D+G235W+N252P+N260Q+N267T+H272V+I278V+S279D+Y281P+K308Q+K309E+K312Q
Q3F+H15Y+D111R+V140I+S163P+S186R+Q215K+S225G+N227T+E232D+G235W+N252P+N260Q+N267T+H272V+I278V+S279D+Y281P+K308Q+K309E+K312Q
Q3F+H15Y+D111R+V140I+S163P+L170G+S186R+Q215K+S225G+N227T+E232D+G235W+N252P+N260Q+N267T+H272V+S279D+Y281P+K308Q+K309E+K312Q
Q3F+H15Y+D111R+V140I+S163P+L170G+S186R+Q215K+S225G+N227T+E232D+G235W+N252P+N260Q+N267T+H272V+I278V+S279D+Y281P+K308Q+K309E+K312Q
Q3F+H15Y+D111R+V140I+S163P+L170G+S186R+S199W+Q215K+S225G+N227T+E232D+G235W+N252P+N260Q+N267T+H272V+S279D+Y281P+K308Q+K309E+K312Q
Q3F+H15Y+V140I+E150N+S163P+S186R+Q215K+S225G+N227T+E232D+G235W+N252P+N260Q+N267T+H272V+S279D+Y281P+K308Q+K309E+K312Q
Q3F+H15Y+V140I+E150N+S163P+S186R+Q215K+S225G+N227T+E232D+G235W+N252P+N260Q+N267T+H272V+I278V+S279D+Y281P+K308Q+K309E+K312Q
Q3F+H15Y+V140I+E150N+S163P+L170G+S186R+Q215K+S225G+N227T+E232D+G235W+N252P+N260Q+N267T+H272V+S279D+Y281P+K308Q+K309E+K312Q
Q3F+H15Y+V140I+E150N+S163P+L170G+S186R+Q215K+S225G+N227T+E232D+G235W+N252P+N260Q+N267T+H272V+I278V+S279D+Y281P+K308Q+K309E+K312Q
Q3F+H15Y+V140I+E150N+S163P+S186R+S199W+Q215K+S225G+N227T+E232D+G235W+N252P+N260Q+N267T+H272V+S279D+Y281P+K308Q+K309E+K312Q
Q3F+H15Y+V140I+S163P+S186R+Q215K+S225G+N227T+E232D+G235W+N252P+N260Q+N267T+H272V+I278V+S279D+Y281P+K308Q+K309E+K312Q
Q3F+H15Y+V140I+S163P+L170G+S186R+Q215K+S225G+N227T+E232D+G235W+N252P+N260Q+N267T+H272V+S279D+Y281P+K308Q+K309E+K312Q
Q3F+H15Y+V140I+S163P+L170G+S186R+Q215K+S225G+N227T+E232D+G235W+N252P+N260Q+N267T+H272V+I278V+S279D+Y281P+K308Q+K309E+K312Q
Q3F+H15Y+V140I+S163P+L170G+S186R+S199W+Q215K+S225G+N227T+E232D+G235W+N252P+N260Q+N267T+H272V+S279D+Y281P+K308Q+K309E+K312Q
Q3F+H15Y+V140I+S163P+L170G+S186R+S199W+Q215K+S225G+N227T+E232D+G235W+N252P+N260Q+N267T+H272V+I278V+S279D+Y281P+K308Q+K309E+K312Q
Q3F+H15Y+V140I+S163P+S186R+S199W+Q215K+S225G+N227T+E232D+G235W+N252P+N260Q+N267T+H272V+I278V+S279D+Y281P+K308Q+K309E+K312Q

The hexosaminidase variants disclosed herein have a sequence identity to the parent hexosaminidase without the substitutions in the variant of at least 60%. The variants may e.g. have a sequence identity to the parent hexosaminidase of at least 65%, at least 70%, at least 75% or at least 80%. The hexosaminidase variants may for example have a sequence identity to the parent hexosaminidase of at least 85%, at least 90%, at least 91%, at least 92%, at least 93%, at least 94% or at least 95%, for example at least 96%, at least 97%, at least 98% or at least 99%, but less than 100%.

In particular, any of the hexosaminidase variants disclosed herein have a sequence identity to SEQ ID NO: 1, SEQ ID NO: 2 or SEQ ID NO: 3 of at least 60%. The hexosaminidase variants may e.g. have a sequence identity to any of SEQ ID NO: 1, 2 or 3 of at least 65%, at least 70%, at least 75% or at least 80%. The hexosaminidase variants may for example have a sequence identity to any of SEQ ID NO: 1, 2 or 3 of at least 85%, at least 90%, at least 91%, at least 92%, at least 93%, at least 94% or at least 95%, for example at least 96%, at least 97% or at least 98%, but less than 100%.

In one aspect, a hexosaminidase variant of the invention has a sequence identity to SEQ ID NO: 2 of at least 90%, at least 91%, at least 92%, at least 93%, at least 94% or at least 95%, for example at least 96%, at least 97%, at least 98% or at least 99%, but less than 100%.

In another aspect, a hexosaminidase variant of the invention has a sequence identity to SEQ ID NO: 3 of at least 90%, at least 91%, at least 92%, at least 93%, at least 94% or at least 95%, for example at least 96%, at least 97%, at least 98% or at least 99%, but less than 100%.

In one aspect, the number of alterations in the variants of the present invention compared to SEQ ID NO: 1, 2 or 3 is 1-20, e.g., 1-10, such as 1, 2, 3, 4, 5, 6, 7, 8, 9 or 10 alterations.

The variants described herein may further comprise an extension of one or more amino acids at the N-terminal and/or C-terminal ends. Alternatively, the variants may further comprise a truncation of one or more amino acids at the N-terminal and/or C-terminal ends.

The present invention thus includes variants as disclosed herein comprising an extension of one or more amino acids at the N-terminal and/or C-terminal ends, or a truncation of one or more amino acids at the N-terminal and/or C-terminal ends, wherein the variants have hexosaminidase activity and have an increased detergent stability compared to the polypeptide of SEQ ID NO: 1, or compared to the polypeptide of SEQ ID NO: 2 or SEQ ID NO: 3.

The variants of the invention may optionally comprise other amino acid changes that may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant molecules are tested for hexosaminidase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide, and/or be inferred from sequence homology and conserved catalytic machinery with a related polypeptide or within a polypeptide or protein family with polypeptides/proteins descending from a common ancestor, typically having similar three-dimensional structures, functions, and significant sequence similarity. Additionally or alternatively, protein structure prediction tools can be used for protein structure modelling to identify essential amino acids and/or active sites of polypeptides. See, for example, Jumper et al., 2021, “Highly accurate protein structure prediction with AlphaFold”, Nature 596: 583-589.

The variants may consist of 300-350 amino acids, e.g., 310 to 340, 315 to 335 or 320 to 330 amino acids.

In an embodiment, the variant has improved stability, in particular improved storage stability, i.e. improved stability under storage in a detergent composition compared to the parent enzyme. As mentioned above, the improved stability may be expressed e.g. by way of a half-life improvement factor (HIF) as described in the examples herein.

In an embodiment, the hexosaminidase variant has an improved stability expressed as half-life improvement factor (HIF) compared to the parent hexosaminidase, i.e. an HIF value greater than 1. Preferably, the variant has half-life improvement factor compared to the parent, e.g. determined as described in the examples herein, of at least 1.2, at least 1.3, at least 1.4, at least 1.5, at least 1.6, at least 1.7, at least 1.8, at least 1.9 or at least 2.0. In preferred embodiments, the variant may have a half-life improvement factor compared to the parent of at least 2.5, at least 3.0, at least 3.5 or at least 4.0.

The hexosaminidase variants of the invention are preferably isolated, more preferably purified, using standard protein purification methods known in the art.

### Preparation of Variants

The present invention also relates to methods for obtaining a variant having hexosaminidase activity, comprising:
(a) introducing into a parent hexosaminidase a substitution at one or more positions, preferably two, three or four positions, corresponding to positions 163, 227, 252 and 309 of SEQ ID NO: 1; and a substitution at one or more positions corresponding to positions 106, 111, 120, 124, 127, 150, 170, 171, 178, 199, 208, 254, 255 and 278 of SEQ ID NO: 1; and
(b) recovering the variant;
wherein the variant has hexosaminidase activity and has at least 60%, e.g., at least 65%, at least 70%, at least 75%, at least 80%, at least 85%, at least 90% or at least 95% sequence identity, but less than 100% sequence identity, to the polypeptide of SEQ ID NO: 1, SEQ ID NO: 2 or SEQ ID NO: 3

Preferably, the method comprises introducing into a parent hexosaminidase, compared to SEQ ID NO: 1, (i) two, three or four substitutions selected from the group consisting of S163P, N227T, N252P and K309E; and (ii) at least one substitution selected from the group consisting of V106M, V106N, D111R, T120V, Y124I, Y124K, Y124L, R127P, E150N, L170G, D171F, D171H, Q178K, S199W, S208W, F254I, T255D and I278V. Preferred substitutions to be introduced into the parent hexosaminidase in (ii) include V106M/N, D111R, T120V, E150N, D171F and I278V, for example at least two of said substitutions. Particularly preferred substitutions include one, two or three of D111R, T120V and E150N.

The method preferably further comprises introducing into the parent hexosaminidase, compared to SEQ ID NO: 1, at least one substitution selected from the group consisting of H15Y, S186R, S225G, E232D, G235W, N260Q, H272V, S279D, Y281P, K308Q and K312Q, for example 2, 3, 4, 5, 6, 7, 8, 9, 10 or all of said substitutions. The method more preferably comprises introducing into the parent hexosaminidase, compared to SEQ ID NO: 1, one or more substitutions selected from the group consisting of Q3I, A49W and N59E, preferably 2 or all 3 of said substitutions; or one or more substitutions selected from the group consisting of Q3F, V140I, Q215K and N267T, preferably 2, 3 or all 4 of said substitutions.

In other embodiments, the method comprises (a) introducing into a parent hexosaminidase any of the sets of substitutions listed above under the heading “Variants”, wherein position numbers are based on the numbering of SEQ ID NO: 1, wherein the variant has a sequence identity of at least 60%, e.g. at least 65%, at least 70%, at least 75%, at least 80%, at least 85%, at least 90% or at least 95%, but less than 100% to the polypeptide of SEQ ID NO: 1, SEQ ID NO: 2 or SEQ ID NO: 3, and wherein the variant has hexosaminidase activity; and (b) recovering the variant.

Preferably, variants obtained by the methods disclosed herein have a sequence identity compared to SEQ ID NO: 2 or SEQ ID NO: 3 of at least 80%, preferably at least 85%, more preferably at least 90%, such as at least 91%, at least 92%, at least 93%, at least 94%, at least 95%, at least 96%, at least 97% or at least 98%.

The variants can be prepared using any mutagenesis procedure known in the art, such as site-directed mutagenesis, synthetic gene construction, semi-synthetic gene construction, random mutagenesis, DNA shuffling, etc.

### Polynucleotides

The present invention also relates to polynucleotides encoding a variant of the present invention.

The polynucleotide may be a genomic DNA, a cDNA, a synthetic DNA, a synthetic RNA, a mRNA, or a combination thereof.

In an aspect, the polynucleotide is isolated.

In another aspect, the polynucleotide is purified.

### Nucleic Acid Constructs

The present invention also relates to nucleic acid constructs comprising a polynucleotide encoding a variant of the present invention operably linked to one or more control sequences that direct the expression of the coding sequence in a suitable host cell under conditions compatible with the control sequences. Examples of control sequences that may be used are promoters, terminators, mRNA stabilizers, leader sequences, polyadenylation sequences, signal peptides, propeptides, regulatory sequences and transcription factors, all of which are well known in the art.

The polynucleotide may be manipulated in a variety of ways to provide for expression of a variant. Manipulation of the polynucleotide prior to its insertion into a vector may be desirable or necessary depending on the expression vector. The techniques for modifying polynucleotides utilizing recombinant DNA methods are well known in the art.

### Expression Vectors

The present invention also relates to recombinant expression vectors comprising a polynucleotide encoding a variant of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the variant at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid. Expression vectors suitable for recombinant expression are well known in the art, as are e.g. methods for introducing them into a host cell.

### Host Cells

The present invention also relates to recombinant host cells, comprising a polynucleotide of the present invention operably linked to one or more control sequences that direct the production of a variant of the present invention.

A construct or vector comprising a polynucleotide is introduced into a host cell so that the construct or vector is maintained as a chromosomal integrant or as a self-replicating extra-chromosomal vector as described earlier. The choice of a host cell will to a large extent depend upon the gene encoding the variant and its source. The recombinant host cell may comprise a single copy, or at least two copies, e.g., three, four, five, or more copies of the polynucleotide of the present invention.

The host cell may be any microbial cell useful in the recombinant production of a polypeptide of the present invention, e.g., a prokaryotic cell or a fungal cell.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

A fungal host cell may be a yeast cell or a filamentous fungal cell.

The filamentous fungal host cell may e.g. be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell. In a preferred embodiment, the filamentous fungal host cell is an Aspergillus, Trichoderma or Fusarium cell. In a further preferred embodiment, the filamentous fungal host cell is an Aspergillus niger, Aspergillus oryzae, Trichoderma reesei, or Fusarium venenatum cell.

In an aspect, the host cell is isolated, preferably purified.

### Methods of Production

The present invention also relates to methods of producing a variant of the present invention, comprising (a) cultivating a recombinant host cell of the invention under conditions conducive for production of the variant; and optionally (b) recovering the variant.

The host cell is cultivated in a nutrient medium suitable for production of the variant using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the variant to be expressed and/or isolated. Suitable media are available from commercial suppliers or may be prepared according to published compositions (e.g., in catalogues of the American Type Culture Collection). If the variant is secreted into the nutrient medium, the variant can be recovered directly from the medium. If the variant is not secreted, it can be recovered from cell lysates.

The variant may be detected using methods known in the art that are specific for the variant, including, but not limited to, the use of specific antibodies, formation of an enzyme product, disappearance of an enzyme substrate, or an enzyme assay determining the relative or specific activity of the variant.

The variant may be recovered from the medium using methods known in the art, including, but not limited to, collection, centrifugation, filtration, extraction, spray-drying, evaporation, or precipitation. In one aspect, the whole fermentation broth is recovered. In another aspect, a cell-free fermentation broth comprising the polypeptide is recovered.

The variant may be purified by a variety of procedures known in the art to obtain substantially pure variants and/or fragments (see, e.g., Wingfield, 2015, Current Protocols in Protein Science; 80(1): 6.1.1-6.1.35; Labrou, 2014, Protein Downstream Processing, 1129: 3-10).

In an alternative aspect, the variant is not recovered from the medium.

### Compositions

The present invention further relates to cleaning compositions comprising at least one hexosaminidase variant according to the invention and at least one cleaning adjunct ingredient. The cleaning composition may for example be used for obtaining an improved deep-cleaning effect, i.e. disruption or removal of a biofilm or components thereof, in items such as textiles, for example for preventing and/or reducing stickiness of an item, for pretreating stains on the item, for preventing and/or reducing redeposition of soil during a wash cycle, for preventing and/or reducing adherence of soil to an item, for maintaining or improving the whiteness of an item and/or for preventing and/or reducing malodor in an item. The hexosaminidase variants of the invention are useful in different types of detergent compositions such as powder and liquid cleaning compositions as well as in e.g. single unit dose compositions.

The composition may contain one or more cleaning adjunct ingredients selected from the group consisting of surfactants, builders, flocculating aid, chelating agents, dye transfer inhibitors, enzymes, enzyme stabilizers, enzyme inhibitors, catalytic materials, bleach activators, hydrogen peroxide, sources of hydrogen peroxide, preformed peracids, polymeric dispersing agents, clay soil removal/anti-redeposition agents, brighteners, suds suppressors, dyes, perfumes, structure elasticizing agents, fabric softeners, carriers, hydrotropes, builders and co-builders, fabric hueing agents, anti-foaming agents, dispersants, processing aids, and/or pigments.

The cleaning composition will typically contain a surfactant and normally other cleaning adjunct ingredients such as a builder or a clay/soil removal/anti-redeposition agent.

The cleaning adjunct ingredient may be one or more enzymes other than a hexosaminidase. The one or more enzymes may e.g. be selected from the group consisting of proteases, amylases, lipases, cutinases, cellulases, DNases, endoglucanases, xyloglucanases, pectinases, pectin lyases, xanthanases, peroxidases, haloperoxygenases, catalases and mannanases. Specific enzymes suitable for the detergent compositions of the invention are described below.

The cleaning composition may be formulated in any suitable form, such as a bar, a homogenous tablet, a tablet having two or more layers, a pouch having one or more compartments, a regular or compact powder, a granule, a paste, a gel, or a regular, compact or concentrated liquid. The cleaning composition can thus e.g. be a liquid detergent or a powder or granular detergent, optionally in “concentrated” or “compact” form. It may also be in the form of a single unit dose composition.

The amount of hexosaminidase in the cleaning composition may vary depending on factors such as the degree of concentration or compactness of the composition and the desired hexosaminidase concentration in the wash liquor. The hexosaminidase will normally be included in the cleaning composition in an amount of up to about 10,000 ppm, typically up to about 5000 ppm or up to about 2000 ppm. The hexosaminidase can e.g. be included in the cleaning composition at a level of from 1 ppm to 10,000 ppm, such as from 10 ppm to 5000 ppm, from 20 ppm to 2000 ppm, from 50 ppm to 1000 ppm, from 80 ppm to 600 ppm, or from 100 ppm to 500 ppm. The unit “ppm” in this context is intended to refer to mg/l for an enzyme added to a liquid composition (e.g. liquid, gel, etc.), or mg/kg for an enzyme added to a solid composition (e.g. powder, granulate, tablet, etc.)

In some aspects, the detergent composition is a liquid or powder laundry detergent, suitable for e.g. washing at high temperature and/or pH, such as at or above 40° C. and/or at or above pH 8. In some aspects, the detergent composition is a liquid or powder laundry detergent, suitable for e.g. washing at low temperature and/or pH, such as at or below 20° C. and/or pH 6. The detergent may also be formulated as a unit dose detergent and/or compact detergent optionally with minimum or no water. The detergent may also be a dishwashing detergent. Such laundry and dishwashing detergents may be phosphate-free.

### Surfactants

A surfactant may be selected among nonionic, anionic and/or amphoteric surfactants, preferably anionic or nonionic surfactants but also amphoteric surfactants may be used. In general, bleach-stable surfactants are preferred. Preferred anionic surfactants are sulphate surfactants and in particular alkyl ether sulphates, especially C-9-15 alcohol ethersulfates, C12-15 primary alcohol ethoxylate, C8-C16 ester sulphates and C10-C14 ester sulphates, such as mono dodecyl ester sulphates Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or salt of fatty acids (soap), and combinations thereof.

The anionic surfactants are preferably added to the detergent in the form of salts. Suitable cations in these salts are alkali metal ions, such as sodium, potassium and lithium and ammonium salts, for example (2-hydroxyethyl) ammonium, bis(2-hydroxyethyl) ammonium and tris(2-hydroxyethyl) ammonium salts. Non-limiting examples of nonionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA), as well as products available under the trade names SPAN and TWEEN, and combinations thereof. Commercially available nonionic surfactants include Plurafac™, Lutensol™ and Pluronic™ from BASF, Dehypon™ series from Cognis and Genapol™ series from Clariant.

### Builders

A builder is preferably selected among phosphates, sodium citrate builders, sodium carbonate, sodium silicate, sodium aluminosilicate (zeolite). Suitable builders are alkali metal or ammonium phosphates, polyphosphates, phosphonates, polyphosphates, carbonates, bicarbonates, borates, citrates, and polycarboxylates. Citrate builders, e.g., citric acid and soluble salts thereof (particularly sodium salt), are polycarboxylate builders. Citrates can be used in combination with zeolite, silicates like the BRITESIL types, and/or layered silicate builders. The builder is preferably added in an amount of about 0-65% by weight, such as about 5% to about 50% by weight. In a laundry detergent, the level of builder is typically about 40-65% by weight, particularly about 50-65% by weight, particularly from 20% to 50% by weight. The builder and/or co-builder may particularly be a chelating agent that forms water-soluble complexes with Ca and Mg. Any builder and/or co-builder known in the art for use in cleaning detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), and (carboxymethyl)inulin (CMI), and combinations thereof. Further non-limiting examples of builders include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycine-N,N-diacetic acid (MGDA), glutamic acid-N,N-diacetic acid (GLDA), 1-hydroxyethane-1,1-diphosphonic acid, N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(sulfomethyl)aspartic acid (SMAS), N-(2-sulfoethyl)-aspartic acid (SEAS), N-(sulfomethylglutamic acid (SMGL), N-(2-sulfoethyl)-glutamic acid (SEGL), N-methyliminodiacetic acid (MIDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA) and N′-(2-hydroxyethyl)ethylenediamine-N,N,N′-triacetic acid (HEDTA), diethanolglycine (DEG), and combinations and salts thereof. Phosphonates suitable for use herein include 1-hydroxyethane-1,1-diphosphonic acid (HEDP), ethylenediaminetetrakis (methylenephosphonicacid) (EDTMPA), diethylenetriaminepentakis (methylenephosphonic acid) (DTMPA or DTPMPA or DTPMP), nitrilotris (methylenephosphonic acid) (ATMP or NTMP), 2-phosphonobutane-1,2,4-tricarboxylic acid (PBTC), hexamethylenediaminetetrakis (methylenephosphonic acid) (HDTMP). The composition may also contain 0-50% by weight, such as about 5% to about 30%, of a detergent co-builder. The detergent composition may include a co-builder alone, or in combination with a builder, for example a zeolite builder. Non-limiting examples of co-builders include homopolymers of polyacrylates or copolymers thereof, such as poly (acrylic acid) (PAA) or copoly (acrylic acid/maleic acid) (PAA/PMA) or polyaspartic acid. Further exemplary builders and/or co-builders are described in, e.g., WO 2009/102854 and U.S. Pat. No. 5,977,053. In some aspects, the builder is a non-phosphorus based builder such as citric acid and/or methylglycine-N, N-diacetic acid (MGDA) and/or glutamic-N, N-diacetic acid (GLDA) and/or salts thereof. The liquid composition may also be phosphate free in that instance the preferred builders includes citrate and/or methylglycine-N, N-diacetic acid (MGDA) and/or glutamic-N, N-diacetic acid (GLDA) and/or salts thereof.

### Bleach Components

The cleaning composition may contain 0-30% by weight, such as about 1% to about 20%, of a bleaching system. Any bleaching system comprising components known in the art for use in cleaning detergents may be utilized. Suitable bleaching system components include sources of hydrogen peroxide; sources of peracids; and bleach catalysts or boosters.

Sources of hydrogen peroxide: Suitable sources of hydrogen peroxide are inorganic persalts, including alkali metal salts such as sodium percarbonate and sodium perborates (usually mono- or tetrahydrate), and hydrogen peroxide-urea (1/1).

Sources of peracids: Peracids may be (a) incorporated directly as preformed peracids or (b) formed in situ in the wash liquor from hydrogen peroxide and a bleach activator (perhydrolysis) or (c) formed in situ in the wash liquor from hydrogen peroxide and a perhydrolase and a suitable substrate for the latter, e.g., an ester.
a) Suitable preformed peracids include, but are not limited to, peroxycarboxylic acids such as peroxybenzoic acid and its ring-substituted derivatives, peroxy-α-naphthoic acid, peroxyphthalic acid, peroxylauric acid, peroxystearic acid, ε-phthalimidoperoxycaproic acid [phthalimidoperoxyhexanoic acid (PAP)], and o-carboxybenzamidoperoxycaproic acid; aliphatic and aromatic diperoxydicarboxylic acids such as diperoxydodecanedioic acid, diperoxyazelaic acid, diperoxysebacic acid, diperoxybrassylic acid, 2-decyldiperoxybutanedioic acid, and diperoxyphthalic, -isophthalic and -terephthalic acids; perimidic acids; peroxymonosulfuric acid; peroxydisulfuric acid; peroxyphosphoric acid; peroxysilicic acid; and mixtures of said compounds. It is understood that the peracids mentioned may in some cases be best added as suitable salts, such as alkali metal salts (e.g., Oxone®) or alkaline earth-metal salts.
b) Suitable bleach activators include those belonging to the class of esters, amides, imides, nitriles or anhydrides and, where applicable, salts thereof. Suitable examples are tetraacetylethylenediamine (TAED), sodium 4-[(3,5,5-trimethylhexanoyl)oxy]benzene-1-sulfonate (ISONOBS), sodium 4-(dodecanoyloxy)benzene-1-sulfonate (LOBS), sodium 4-(decanoyloxy)benzene-1-sulfonate, 4-(decanoyloxy)benzoic acid (DOBA), sodium 4-(nonanoyloxy)benzene-1-sulfonate (NOBS), and/or those disclosed in WO98/17767. A particular family of bleach activators of interest was disclosed in EP624154 and particularly preferred in that family is acetyl triethyl citrate (ATC). ATC or a short chain triglyceride like triacetin has the advantage that they are environmentally friendly. Furthermore, acetyl triethyl citrate and triacetin have good hydrolytical stability in the product upon storage and are efficient bleach activators. Finally, ATC is multifunctional, as the citrate released in the perhydrolysis reaction may function as a builder.

Bleach catalysts and boosters: The bleaching system may also include a bleach catalyst or booster. Some non-limiting examples of bleach catalysts that may be used in the compositions of the present invention include manganese oxalate, manganese acetate, manganese-collagen, cobalt-amine catalysts and manganese triazacyclononane (MnTACN) catalysts; particularly preferred are complexes of manganese with 1,4,7-trimethyl-1,4,7-triazacyclononane (Me3-TACN) or 1,2,4,7-tetramethyl-1,4,7-triazacyclononane (Me4-TACN), in particular Me3-TACN, such as the dinuclear manganese complex [(Me3-TACN)Mn(O)3Mn(Me3-TACN)](PF6)2, and [2,2′,2″-nitrilotris(ethane-1,2-diylazanylylidene-KN-methanylylidene)triphenolato-κ3O]manganese(III). The bleach catalysts may also be other metal compounds, such as iron or cobalt complexes. Other suitable bleach catalysts are acylhydrazone catalysts such as those disclosed in US 2014/0323381.

In some aspects, where a source of a peracid is included, an organic bleach catalyst or bleach booster may be used having one of the following formulae:

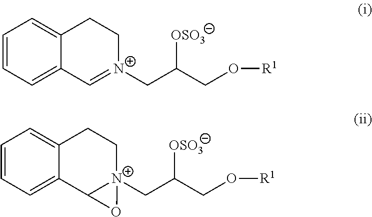

(iii) and mixtures thereof; wherein each R1 is independently a branched alkyl group containing from 9 to 24 carbons or linear alkyl group containing from 11 to 24 carbons, preferably each R1 is independently a branched alkyl group containing from 9 to 18 carbons or linear alkyl group containing from 11 to 18 carbons, more preferably each R1 is independently selected from the group consisting of 2-propylheptyl, 2-butyloctyl, 2-pentylnonyl, 2-hexyldecyl, dodecyl, tetradecyl, hexadecyl, octadecyl, isononyl, isodecyl, isotridecyl and isopentadecyl.

Other exemplary bleaching systems are described, e.g. in WO 2007/087258, WO 2007/087244, WO 2007/087259, EP 1867708 (Vitamin K) and WO 2007/087242. Suitable photobleaches may for example be sulfonated zinc or aluminium phthalocyanines.

The choice of detergent components may include, for textile care, the consideration of the type of textile to be cleaned, the type and/or degree of soiling, the temperature at which cleaning is to take place, and the formulation of the detergent product. Although components mentioned below are categorized by general header according to a functionality, this is not to be construed as a limitation, as a component may comprise additional functionalities as will be appreciated by the skilled artisan, including the exemplary non-limiting components shown in below.

### Hydrotropes

The detergent composition may contain 0-10% by weight, for example 0-5% by weight, such as about 0.5 to about 5%, or about 3% to about 5%, of a hydrotrope. Any hydrotrope known in the art for use in detergents may be utilized. Non-limiting examples of hydrotropes include sodium benzenesulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

### Polymers

The detergent composition may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fibre protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575. Salts of the above-mentioned polymers are also contemplated.

### Fabric Hueing Agents

The detergent composition of the present invention may also include fabric hueing agents such as dyes or pigments, which when formulated in detergent compositions can deposit onto a fabric when said fabric is contacted with a wash liquor comprising said detergent compositions and thus altering the tint of said fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO 2005/03274, WO 2005/03275, WO 2005/03276 and EP 1876226 (hereby incorporated by reference). The detergent composition preferably comprises from about 0.00003 wt % to about 0.2 wt %, from about 0.00008 wt % to about 0.05 wt %, or even from about 0.0001 wt % to about 0.04 wt % fabric hueing agent. The composition may comprise from 0.0001 wt % to 0.2 wt % fabric hueing agent, this may be especially preferred when the composition is in the form of a unit dose pouch. Suitable hueing agents are also disclosed in, e.g. WO 2007/087257 and WO 2007/087243.

### Enzymes

The detergent composition may comprise one or more additional enzymes such as a protease, lipase, cutinase, amylase, DNase, carbohydrase, cellulase, pectinase, mannanase, arabinase, galactanase, xylanase, hexosaminidase, oxidase, e.g., a laccase, and/or peroxidase.

In general, the properties of the selected enzyme(s) should be compatible with the selected detergent, (i.e., pH-optimum, compatibility with other enzymatic and non-enzymatic ingredients, etc.), and the enzyme(s) should be present in effective amounts.

### Cellulases

Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. Nos. 4,435,307, 5,648,263, 5,691,178, 5,776,757 and WO 89/09259.

Especially suitable cellulases are the alkaline or neutral cellulases having colour care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. Nos. 5,457,046, 5,686,593, 5,763,254, WO 95/24471, WO 98/12307 and WO 99/001544.

Other cellulases are endo-beta-1,4-glucanase enzyme having a sequence of at least 97% identity to the amino acid sequence of position 1 to position 773 of SEQ ID NO: 2 of WO 2002/099091 or a family 44 xyloglucanase, which a xyloglucanase enzyme having a sequence of at least 60% identity to positions 40-559 of SEQ ID NO: 2 of WO 2001/062903.

Commercially available cellulases include Celluzyme™, and Carezyme™ (Novozymes A/S) Carezyme Premium™ (Novozymes A/S), Celluclean™ (Novozymes A/S), Celluclean Classic™ (Novozymes A/S), Cellusoft™ (Novozymes A/S), Whitezyme™ (Novozymes A/S), Clazinase™, and Puradax HA™ (Genencor International Inc.), and KAC-500(B)™ (Kao Corporation).

### Proteases

Suitable proteases may be of any origin, but are preferably of bacterial or fungal origin, optionally in the form of protein engineered or chemically modified mutants. The protease may be an alkaline protease, such as a serine protease or a metalloprotease. A serine protease may for example be of the S1 family, such as trypsin, or the S8 family such as a subtilisin. A metalloprotease may for example be a thermolysin, e.g. from the M4 family, or another metalloprotease such as those from the M5, M7 or M35 families.

The term “subtilases” refers to a sub-group of serine proteases according to Siezen et al., Protein Eng. 4 (1991) 719-737 and Siezen et al., Protein Sci. 6 (1997) 501-523. Serine proteases are a subgroup of proteases characterized by having a serine in the active site, which forms a covalent adduct with the substrate. The subtilases may be divided into six subdivisions, the Subtilisin family, the Thermitase family, the Proteinase K family, the Lantibiotic peptidase family, the Kexin family and the Pyrolysin family.

Although proteases suitable for detergent use may be obtained from a variety of organisms, including fungi such as Aspergillus, detergent proteases have generally been obtained from bacteria and in particular from Bacillus. Examples of Bacillus species from which subtilases have been derived include Bacillus lentus, Bacillus alkalophilus, Bacillus subtilis, Bacillus amyloliquefaciens, Bacillus licheniformis, Bacillus pumilus and Bacillus gibsonii. Particular subtilisins include subtilisin lentus, subtilisin Novo, subtilisin Carlsberg, subtilisin BPN′, subtilisin 309, subtilisin 147 and subtilisin 168 and e.g. protease PD138 (described in WO 93/18140). Other useful proteases are e.g. those described in WO 01/16285 and WO 02/16547.

Examples of trypsin-like proteases include the Fusarium protease described in WO 94/25583 and WO 2005/040372, and the chymotrypsin proteases derived from Cellumonas described in WO 2005/052161 and WO 2005/052146.

Examples of metalloproteases include the neutral metalloproteases described in WO 2007/044993 such as those derived from Bacillus amyloliquefaciens, as well as e.g. the metalloproteases described in WO 2015/158723 and WO 2016/075078.

Examples of useful proteases are the protease variants described in WO 89/06279 WO 92/19729, WO 96/34946, WO 98/20115, WO 98/20116, WO 99/11768, WO 01/44452, WO 03/006602, WO 2004/003186, WO 2004/041979, WO 2007/006305, WO 2011/036263, WO 2014/207227, WO 2016/087617 and WO 2016/174234. Preferred protease variants may, for example, comprise one or more of the mutations selected from the group consisting of: S3T, V41, S9R, S9E, A15T, S24G, S24R, K27R, N42R, S55P, G59E, G59D, N60D, N60E, V66A, N74D, S85R, A96S, S97G, S97D, S97A, S97SD, S99E, S99D, S99G, S99M, S99N, S99R, S99H, S101A, V102I, V102Y, V102N, S104A, G116V, G116R, H118D, H118N, A120S, S126L, P127Q, S128A, S154D, A156E, G157D, G157P, S158E, Y161A, R164S, Q176E, N179E, S182E, Q185N, A188P, G189E, V193M, N198D, V199I, Q200L, Y203W, S206G, L211Q, L211D, N212D, N212S, M216S, A226V, K229L, Q230H, Q239R, N246K, S253D, N255W, N255D, N255E, L256E, L256D T268A and R269H, wherein position numbers correspond to positions of the Bacillus lentus protease shown in SEQ ID NO: 1 of WO 2016/001449. Protease variants having one or more of these mutations are preferably variants of the Bacillus lentus protease (Savinase®, also known as subtilisin 309) shown in SEQ ID NO: 1 of WO 2016/001449 or of the Bacillus amyloliquefaciens protease (BPN′) shown in SEQ ID NO: 2 of WO 2016/001449. Such protease variants preferably have at least 80% sequence identity to SEQ ID NO: 1 or to SEQ ID NO: 2 of WO 2016/001449.

Another protease of interest is the alkaline protease from Bacillus lentus DSM 5483, as described for example in WO 91/02792, and variants thereof which are described for example in WO 92/21760, WO 95/23221, EP 1921147, EP 1921148 and WO 2016/096711.

The protease may alternatively be a variant of the TY145 protease having SEQ ID NO: 1 of WO 2004/067737, for example a variant comprising a substitution at one or more positions corresponding to positions 27, 109, 111, 171, 173, 174, 175, 180, 182, 184, 198, 199 and 297 of SEQ ID NO: 1 of WO 2004/067737, wherein said protease variant has a sequence identity of at least 75% but less than 100% to SEQ ID NO: 1 of WO 2004/067737. TY145 variants of interest are described in e.g. WO 2015/014790, WO 2015/014803, WO 2015/014804, WO 2016/097350, WO 2016/097352, WO 2016/097357 and WO 2016/097354.

Suitable commercially available protease enzymes include those sold under the trade names Alcalase®, Duralase™, Durazym™, Relase®, Relase® Ultra, Savinase®, Savinase® Ultra, Primase™, Polarzyme®, Kannase®, Liquanase®, Liquanase® Ultra, Ovozyme®, Coronase®, Coronase® Ultra, Blaze®, Blaze Evity® 100T, Blaze Evity® 125T, Blaze Evity® 150T, Blaze Evity® 200T, Neutrase®, Everlase®, Esperase®, Progress® Uno, Progress® In, Progress® Key and Progress® Excel (Novozymes A/S), those sold under the tradename Maxatase™, Maxacal™, Maxapem®, Purafect® Ox, Purafect® OxP, Puramax®, FN2™, FN3™, FN4^{ex}™, Excellase®, Excellenz™ P1000, Excellenz™ P1250, Eraser™, Preferenz® P100, Preferenz® P300, Purafect Prime, Preferenz P110™, Effectenz P1000™, Purafect®, Effectenz P1050™, Purafect® Ox, Effectenz™ P2000, Purafast™, Properase®, Opticlean™ and Optimase® (Danisco/DuPont), BLAP (sequence shown in FIG. 29 of U.S. Pat. No. 5,352,604) and variants hereof (Henkel AG), and KAP (Bacillus alkalophilus subtilisin) from Kao.

### Lipases and Cutinases

Suitable lipases and cutinases include those of bacterial or fungal origin. Chemically modified or protein engineered mutant enzymes are included. Examples include lipase from Thermomyces, e.g. from T. lanuginosus (previously named Humicola lanuginosa) as described in EP258068 and EP305216, cutinase from Humicola, e.g. H. insolens (WO96/13580), lipase from strains of Pseudomonas (some of these now renamed to Burkholderia), e.g. P. alcaligenes or P. pseudoalcaligenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), Geobacillus stearothermophilus lipase (WO11/084417), lipase from Bacillus subtilis (WO11/084599), and lipase from Streptomyces griseus (WO11/150157) and S. pristinaespiralis (WO12/137147).

Other examples are lipase variants such as those described in EP407225, WO92/05249, WO94/01541, WO94/25578, WO95/14783, WO95/30744, WO95/35381, WO95/22615, WO96/00292, WO97/04079, WO97/07202, WO00/34450, WO00/60063, WO01/92502, WO07/87508 and WO09/109500.

Preferred commercial lipase products include Lipolase™, Lipex™; Lipolex™ and Lipoclean™ (Novozymes A/S), Lumafast (originally from Genencor) and Lipomax (originally from Gist-Brocades).

Still other examples are lipases sometimes referred to as acyltransferases or perhydrolases, e.g. acyltransferases with homology to Candida antarctica lipase A (WO10/111143), acyltransferase from Mycobacterium smegmatis (WO05/56782), perhydrolases from the CE 7 family (WO09/67279), and variants of the M. smegmatis perhydrolase in particular the S54V variant used in the commercial product Gentle Power Bleach from Huntsman Textile Effects Pte Ltd (WO10/100028).

### Amylases

Suitable amylases which can be used together with the hexosaminidase s of the invention may be an alpha-amylase or a glucoamylase and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Suitable amylases include amylases having SEQ ID NO: 2 in WO 95/10603 or variants having 90% sequence identity to SEQ ID NO: 1 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylase comprising residues 1-33 of the alpha-amylase obtained from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, 1201, A209 and Q264. Most preferred variants of the hybrid alpha-amylase comprising residues 1-33 of the alpha-amylase obtained from B. amyloliquefaciens shown in SEQ ID NO: 6 of WO 2006/066594 and residues 36-483 of SEQ ID NO: 4 are those having the substitutions:
M197T;
H156Y+A181T+N190F+A209V+Q264S; or
G48A+T491+G107A+H156Y+A181T+N190F+I201F+A209V+Q264S.

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, I206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476, using SEQ ID NO: 2 of WO 96/023873 for numbering. More preferred variants are those having a deletion in two positions selected from 181, 182, 183 and 184, such as 181 and 182, 182 and 183, or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further suitable amylases are amylases having SEQ ID NO: 2 of WO 09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T165I, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:
N128C+K178L+T182G+Y305R+G475K;
N128C+K178L+T182G+F202Y+Y305R+D319T+G475K;
S125A+N128C+K178L+T182G+Y305R+G475K; or
S125A+N128C+T131I+T165I+K178L+T182G+Y305R+G475K wherein the variants are C-terminally truncated and optionally further comprises a substitution at position 243 and/or a deletion at position 180 and/or position 181.

Other suitable amylases are the alpha-amylase having SEQ ID NO: 12 in WO 01/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO 2011/098531, WO 2013/001078 and WO 2013/001087.

Commercially available amylases include Duramyl™, Termamyl™, Fungamyl™, Stainzyme™, Stainzyme Plus™, Natalase™, Liquozyme X and BAN™ (from Novozymes A/S), and Rapidase™, Purastar™/Effectenz™, Powerase and Preferenz S100 (from Genencor International Inc./DuPont).

### DNases

The term “DNase” means a polypeptide having DNase (deoxyribonuclease) activity that catalyzes the hydrolytic cleavage of phosphodiester linkages in a DNA backbone, thus degrading DNA. DNase polypeptides have been found to be useful for deep cleaning of microbial biofilm that may be present on surfaces such as textiles or dishware or other hard surfaces, and which consists of a matrix of extracellular polymeric substance (EPS) composed of extracellular DNA, proteins, and polysaccharides.

The DNase polypeptide is typically a microbial enzyme, preferably of fungal or bacterial origin, or a genetically engineered variant of a microbial DNase.

Suitable bacterial DNases may, for example, be obtained from species of Bacillus and related genera (cf. Patel and Gupta, Int. J. Syst. Evol. Microbiol. 2020; 70:406-438, who proposed six new Bacillaceae genera from species formerly classified as belonging to the genus Bacillus), e.g. from Bacillus, Cytobacillus, Metabacillus, Alkalihalobacillus, Rossellomorea or Mesobacillus. Examples of species from which DNases may be obtained include Bacillus licheniformis, Bacillus subtilis, Bacillus horikoshii, Cytobacillus horneckiae, Metabacillus indicus, Alkalihalobacillus algicola, Rossellomorea vietnamensis, Alkalihalobacillus hwajinpoensis, Metabacillus indicus, Mesobacillus campisalis, Bacillus idriensis, Bacillus algicola, Bacillus marisflavi and Bacillus luciferensis. Preferred bacterial DNases include those obtained from Metabacillus indicus (previously known as Bacillus cibi) and variants thereof.

DNases may also be obtained from a fungal species. Examples of preferred fungal DNases are those obtained from Aspergillus, for example from Aspergillus oryzae, from Trichoderma, for example from Trichoderma harzianum, from Vibressa, for example from Vibressea flavovirens, from Morchella, for example from Morchella costata, and from Rhizoctonia, for example from Rhizoctonia solani, as well as variants thereof. Preferred fungal DNases include those obtained from Aspergillus oryzae and variants thereof.

Suitable DNases, DNase variants, and use thereof in detergent compositions are disclosed, for example, in WO 2014/087011, WO 2015/155350, WO 2015/155351, WO 2017/060475, WO 2017/060493, WO 2017/060505, WO 2017/064269, WO 2018/011277, WO 2018/177203, WO 2018/177936, WO 2018/177938, WO 2019/081724, WO 2019/081721, WO 2021/130167, WO 2022/194668 and WO 2022/194673.

### Peroxidases/Oxidases

A peroxidase may be comprised by the enzyme classification EC 1.11.1.7, as set out by the Nomenclature Committee of the International Union of Biochemistry and Molecular Biology (IUBMB), or any fragment obtained therefrom, exhibiting peroxidase activity. Suitable peroxidases include those of plant, bacterial or fungal origin. Chemically modified or protein engineered mutants are included. Examples of useful peroxidases include peroxidases from Coprinopsis, e.g., from C. cinerea (EP 179,486), and variants thereof as those described in WO 93/24618, WO 95/10602, and WO 98/15257. A peroxidase may also include a haloperoxidase enzyme, such as chloroperoxidase, bromoperoxidase and compounds exhibiting chloroperoxidase or bromoperoxidase activity. Haloperoxidases are classified according to their specificity for halide ions. Chloroperoxidases (E.C. 1.11.1.10) catalyze formation of hypochlorite from chloride ions. In an aspect, the haloperoxidase is a chloroperoxidase. Preferably, the haloperoxidase is a vanadium haloperoxidase, i.e., a vanadate-containing haloperoxidase. In a preferred method the vanadate-containing haloperoxidase is combined with a source of chloride ion. Haloperoxidases have been isolated from many different fungi, in particular from the fungus group dematiaceous hyphomycetes, such as Caldariomyces, e.g., C. fumago, Alternaria, Curvularia, e.g., C. verruculosa and C. inaequalis, Drechslera, Ulocladium and Botrytis. Haloperoxidases have also been isolated from bacteria such as Pseudomonas, e.g., P. pyrrocinia and Streptomyces, e.g., S. aureofaciens. In a preferred aspect, the haloperoxidase is derivable from Curvularia sp., in particular Curvularia verruculosa or Curvularia inaequalis, such as C. inaequalis CBS 102.42 as described in WO 95/27046; or C. verruculosa CBS 147.63 or C. verruculosa CBS 444.70 as described in WO 97/04102; or from Drechslera hartlebii as described in WO 01/79459, Dendryphiella salina as described in WO 01/79458, Phaeotrichoconis crotalarie as described in WO 01/79461, or Geniculosporium sp. as described in WO 01/79460.

An oxidase includes any laccase enzyme comprised by the enzyme classification EC 1.10.3.2, or any fragment obtained therefrom exhibiting laccase activity, or a compound exhibiting a similar activity, such as a catechol oxidase (EC 1.10.3.1), an o-aminophenol oxidase (EC 1.10.3.4), or a bilirubin oxidase (EC 1.3.3.5).

Preferred laccase enzymes are enzymes of microbial origin. The enzymes may be obtained from plants, bacteria or fungi (including filamentous fungi and yeasts).

Suitable examples from fungi include a laccase derivable from a strain of Bacillus, Neurospora, e.g., N. crassa, Podospora, Botrytis, Collybia, Fomes, Lentinus, Pleurotus, Trametes, e.g., T. villosa and T. versicolor, Rhizoctonia, e.g., R. solani, Coprinopsis, e.g., C. cinerea, C. comatus, C. friesii, and C. plicatilis, Psathyrella, e.g., P. condelleana, Panaeolus, e.g., P. papilionaceus, Myceliophthora, e.g., M. thermophila, Schytalidium, e.g., S. thermophilum, Polyporus, e.g., P. pinsitus, Phlebia, e.g., P. radiata (WO 92/01046), or Coriolus, e.g., C. hirsutus (JP 2238885).

Suitable examples from bacteria include a laccase derivable from a strain of Bacillus.

A laccase obtained from Coprinopsis or Myceliophthora is preferred; a laccase obtained from Coprinopsis cinerea, as disclosed in WO 97/08325; or from Myceliophthora thermophila, as disclosed in WO 95/33836.

The detergent enzyme(s) may be included in a detergent composition by adding separate additives containing one or more enzymes, or by adding a combined additive comprising these enzymes. A detergent additive of the invention, i.e., a separate additive or a combined additive, can be formulated, for example, as a granulate, liquid, slurry, etc. Preferred detergent additive formulations are granulates, non-dusting granulates, liquids, stabilized liquids and slurries.

Non-dusting granulates may be produced, e.g. as disclosed in U.S. Pat. Nos. 4,106,991 and 4,661,452 and may optionally be coated by methods known in the art. Examples of waxy coating materials are poly (ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591. Liquid enzyme preparations may, for instance, be stabilized by adding a polyol such as propylene glycol, a sugar or sugar alcohol, lactic acid or boric acid according to established methods. Protected enzymes may be prepared according to the method disclosed in EP 238216.

### Dispersants

The detergent compositions of the present invention can also contain dispersants. Powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc.

### Dye Transfer Inhibiting Agents

The cleaning compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

### Fluorescent Whitening Agents

The detergent composition may preferably also contain additional components that may tint articles being cleaned, such as fluorescent whitening agent or optical brighteners. Where present the brightener is preferably at a level of about 0.01% to about 0.5%. Any fluorescent whitening agent suitable for use in a laundry detergent composition may be used in the composition of the present invention. The most commonly used fluorescent whitening agents are those belonging to the classes of diaminostilbene-sulfonic acid derivatives, diarylpyrazoline derivatives and bisphenyl-distyryl derivatives. Examples of the diaminostilbene-sulfonic acid derivative type of fluorescent whitening agents include the sodium salts of: 4,4′-bis-(2-diethanolamino-4-anilino-s-triazin-6-ylamino) stilbene-2,2′-disulfonate, 4,4′-bis-(2,4-dianilino-s-triazin-6-ylamino) stilbene-2.2′-disulfonate, 4,4′-bis-(2-anilino-4-(N-methyl-N-2-hydroxy-ethylamino)-s-triazin-6-ylamino) stilbene-2,2′-disulfonate, 4,4′-bis-(4-phenyl-1,2,3-triazol-2-yl)stilbene-2,2′-disulfonate and sodium 5-(2H-naphtho[1,2-d][1,2,3]triazol-2-yl)-2-[(E)-2-phenylvinyl]benzenesulfonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4-anilino-s-triazin-6-ylamino) stilbene-2,2′-disulfonate. Tinopal CBS is the disodium salt of 2,2′-bis-(phenyl-styryl)-disulfonate. Also preferred are fluorescent whitening agents is the commercially available Parawhite KX, supplied by Paramount Minerals and Chemicals, Mumbai, India. Tinopal CBS-X is a 4.4′-bis-(sulfostyryl)-biphenyl disodium salt also known as disodium distyrylbiphenyl disulfonate. Other fluorescers suitable for use in the invention include the 1-3-diaryl pyrazolines and the 7-alkylaminocoumarins.

Suitable fluorescent brightener levels include lower levels of from about 0.01, from 0.05, from about 0.1 or even from about 0.2 wt % to upper levels of 0.5 or even 0.75 wt %.

### Soil Release Polymers

The detergent compositions may also include one or more soil release polymers which aid the removal of soils from fabrics such as cotton and polyester based fabrics, the removal of hydrophobic soils from polyester based fabrics. The soil release polymers may for example be nonionic or anionic terephthalte based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers is amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore, random graft co-polymers are suitable soil release polymers. Suitable graft co-polymers are described in more detail in WO 2007/138054, WO 2006/108856 and WO 2006/113314 (hereby incorporated by reference). Other soil release polymers are substituted polysaccharide structures especially substituted cellulosic structures such as modified cellulose deriviatives such as those described in EP 1867808 or WO 2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

### Anti-Redeposition Agents

The detergent compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

### Rheology Modifiers

The detergent compositions of the present invention may also include one or more rheology modifiers, structurants or thickeners, as distinct from viscosity reducing agents. The rheology modifiers are selected from the group consisting of non-polymeric crystalline, hydroxy-functional materials, polymeric rheology modifiers which impart shear thinning characteristics to the aqueous liquid matrix of a liquid detergent composition. The rheology and viscosity of the detergent can be modified and adjusted by methods known in the art, for example as shown in EP 2169040.

Other suitable adjunct materials include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, and structurants for liquid detergents and/or structure elasticizing agents.

### Other Materials

Any detergent components known in the art for use in the cleaning composition of the invention may also be utilized. Other optional detergent components include anti-corrosion agents, anti-shrink agents, anti-soil redeposition agents, anti-wrinkling agents, bactericides, binders, corrosion inhibitors, disintegrants/disintegration agents, dyes, enzyme stabilizers (including boric acid, borates, CMC, and/or polyols such as propylene glycol), fabric conditioners including clays, fillers/processing aids, fluorescent whitening agents/optical brighteners, foam boosters, foam (suds) regulators, perfumes, soil-suspending agents, softeners, suds suppressors, tarnish inhibitors, and wicking agents, either alone or in combination. Any ingredient known in the art for use in detergents may be utilized. The choice of such ingredients is well within the skill of the artisan.

### Formulation of Detergent Products

The detergent composition may be in any convenient form, e.g., a bar, a homogenous tablet, a tablet having two or more layers, a regular or compact powder, a granule, a paste, a gel, or a regular, compact or concentrated liquid. Other detergent formulation forms include single unit dose forms such as layered forms and pouches.

Pouches can be configured as single or multicompartments. They can be of any form, shape and material which is suitable for hold the composition, e.g. without allowing release of the composition from the pouch prior to water contact. The pouch is made from water soluble film which encloses an inner volume, which can be divided into compartments. Preferred films are polymeric materials, preferably polymers which are formed into a film or sheet. Preferred polymers, copolymers or derivates thereof are selected from polyacrylates and water-soluble acrylate copolymers, methyl cellulose, carboxy methyl cellulose, sodium dextrin, ethyl cellulose, hydroxyethyl cellulose, hydroxypropyl methyl cellulose, maltodextrin, polymethacrylates, most preferably polyvinyl alcohol copolymers and hydroxypropyl methyl cellulose (HPMC). Preferably the level of polymer in a film such as is at least about 60%. Preferred average molecular weight will typically be about 20,000 to about 150,000. Films can also be of blend compositions comprising hydrolytically degradable and water-soluble polymer blends such as polylactide and polyvinyl alcohol plus plasticisers such as glycerol, ethylene glycerol, propylene glycol, sorbitol and mixtures thereof. The pouches can comprise a solid laundry cleaning composition or part components and/or a liquid cleaning composition or part components separated by the water-soluble film. Compartments for liquid components can be different in composition than compartments containing solids; see e.g. US 2009/0011970 A1.

Detergent ingredients can be separated physically from each other by compartments in water dissolvable pouches or in different layers of tablets, thereby avoiding negative storage interaction between components. Different dissolution profiles of each of the compartments can also give rise to delayed dissolution of selected components in the wash solution.

A liquid or gel detergent which is not unit dosed may be aqueous, typically containing at least 20% by weight and up to 95% water, such as up to about 70% water, up to about 65% water, up to about 55% water, up to about 45% water, or up to about 35% water. Concentrated liquid detergents may have lower water contents, for example not more than about 30% or not more than about 20%, e.g. in the range of about 1% to about 20%, such as from about 2% to about 15%. Other types of liquids, including without limitation, alkanols, amines, diols, ethers and polyols may be included in an aqueous liquid or gel. An aqueous liquid or gel detergent may contain from 0-30% organic solvent. A liquid or gel detergent may alternatively be non-aqueous.

Liquid detergent compositions may be formulated to have a moderate pH of e.g. from about 6 to about 10, such as about pH 7, about pH 8 or about pH 9, or they may be formulated to have a higher pH of e.g. from about 10 to about 12, such as about pH 10, about pH 11 or about pH 12.

Unless indicated otherwise, the term “liquid” as used herein should be understood to encompass any kind of liquid detergent composition, for example concentrated liquids, gels, or the liquid or gel part of e.g. a pouch with one or more compartments.

### Granular Detergent Formulations

Enzymes in the form of granules, comprising an enzyme-containing core and optionally one or more coatings, are commonly used in granular (powder) detergents. Various methods for preparing the core are well-known in the art and include, for example, a) spray drying of a liquid enzyme-containing solution, b) production of layered products with an enzyme coated as a layer around a pre-formed inert core particle, e.g. using a fluid bed apparatus, c) absorbing an enzyme onto and/or into the surface of a pre-formed core, d) extrusion of an enzyme-containing paste, e) suspending an enzyme-containing powder in molten wax and atomization to result in prilled products, f) mixer granulation by adding an enzyme-containing liquid to a dry powder composition of granulation components, g) size reduction of enzyme-containing cores by milling or crushing of larger particles, pellets, etc., and h) fluid bed granulation. The enzyme-containing cores may be dried, e.g. using a fluid bed drier or other known methods for drying granules in the feed or enzyme industry, to result in a water content of typically 0.1-10% w/w water.

The enzyme-containing cores are optionally provided with a coating to improve storage stability and/or to reduce dust formation. One type of coating that is often used for enzyme granulates for detergents is a salt coating, typically an inorganic salt coating, which may e.g. be applied as a solution of the salt using a fluid bed. Other coating materials that may be used are, for example, polyethylene glycol (PEG), methyl hydroxy-propyl cellulose (MHPC) and polyvinyl alcohol (PVA). The granules may contain more than one coating, for example a salt coating followed by an additional coating of a material such as PEG, MHPC or PVA.

For further information on enzyme granules and production thereof, see WO 2013/007594 as well as e.g. WO 2009/092699, EP 1705241, EP 1382668, WO 2007/001262, U.S. Pat. No. 6,472,364, WO 2004/074419 and WO 2009/102854.

### Formulation of Enzyme in Co-Granule

The hexosaminidase may be formulated as a granule for example as a co-granule that combines one or more enzymes. Each enzyme will then be present in more granules securing a more uniform distribution of enzymes in the detergent. This also reduces the physical segregation of different enzymes due to different particle sizes. Methods for producing multi-enzyme co-granulate for the detergent industry is disclosed in the IP.com disclosure IPCOM000200739D.

Another example of formulation of enzymes using co-granulates are disclosed in WO 2013/188331, which relates to a detergent composition comprising (a) a multi-enzyme co-granule; (b) less than 10 wt zeolite (anhydrous basis); and (c) less than 10 wt phosphate salt (anhydrous basis), wherein said enzyme co-granule comprises from 10 to 98 wt % moisture sink components and the composition additionally comprises from 20 to 80 wt % detergent moisture sink components. WO 2013/188331 also relates to a method of treating and/or cleaning a surface, preferably a fabric surface comprising the steps of (i) contacting said surface with the detergent composition as claimed and described herein aqueous wash liquor, (ii) rinsing and/or drying the surface.

### Liquid Formulations

The present invention also relates to liquid compositions comprising a hexosaminidase variant of the invention. The composition may comprise an enzyme stabilizer (examples of which include polyols such as propylene glycol or glycerol, sugar or sugar alcohol, lactic acid, reversible protease inhibitor, boric acid, or a boric acid derivative, e.g., an aromatic borate ester, or a phenyl boronic acid derivative such as 4-formylphenyl boronic acid).

In some embodiments, fillers or carrier materials are included to increase the volume of such compositions. Suitable filler or carrier materials include, but are not limited to, various salts of sulfate, carbonate and silicate as well as talc, clay and the like. Suitable filler or carrier materials for liquid compositions include, but are not limited to, water or low molecular weight primary and secondary alcohols including polyols and diols. Examples of such alcohols include, but are not limited to, methanol, ethanol, propanol and isopropanol. In some embodiments, the compositions contain from about 5% to about 90% of such materials.

In an aspect, the liquid formulation comprises 20-80% w/w of polyol. In one embodiment, the liquid formulation comprises 0.001-2% w/w preservative.

In another embodiment, the invention relates to liquid formulations comprising:
(A) 0.001-25% w/w of a variant of the present invention;
(B) 20-80% w/w of polyol;
(C) optionally 0.001-2% w/w preservative; and
(D) water.

In another embodiment, the invention relates to liquid formulations comprising:
(A) 0.001-25% w/w of a variant of the present invention;
(B) 0.001-2% w/w preservative;
(C) optionally 20-80% w/w of polyol; and
(D) water.

In another embodiment, the liquid formulation comprises one or more formulating agents, such as a formulating agent selected from the group consisting of polyol, sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch, PVA, acetate and phosphate, preferably selected from the group consisting of sodium sulfate, dextrin, cellulose, sodium thiosulfate, kaolin and calcium carbonate. In one embodiment, the polyols is selected from the group consisting of glycerol, sorbitol, propylene glycol (MPG), ethylene glycol, diethylene glycol, triethylene glycol, 1,2-propylene glycol or 1,3-propylene glycol, dipropylene glycol, polyethylene glycol (PEG) having an average molecular weight below about 600 and polypropylene glycol (PPG) having an average molecular weight below about 600, more preferably selected from the group consisting of glycerol, sorbitol and propylene glycol (MPG) or any combination thereof.

In another embodiment, the liquid formulation comprises 20-80% polyol (i.e., total amount of polyol), e.g., 25-75% polyol, 30-70% polyol, 35-65% polyol, or 40-60% polyol. In one embodiment, the liquid formulation comprises 20-80% polyol, e.g., 25-75% polyol, 30-70% polyol, 35-65% polyol, or 40-60% polyol, wherein the polyol is selected from the group consisting of glycerol, sorbitol, propylene glycol (MPG), ethylene glycol, diethylene glycol, triethylene glycol, 1,2-propylene glycol or 1,3-propylene glycol, dipropylene glycol, polyethylene glycol (PEG) having an average molecular weight below about 600 and polypropylene glycol (PPG) having an average molecular weight below about 600. In one embodiment, the liquid formulation comprises 20-80% polyol (i.e., total amount of polyol), e.g., 25-75% polyol, 30-70% polyol, 35-65% polyol, or 40-60% polyol, wherein the polyol is selected from the group consisting of glycerol, sorbitol and propylene glycol (MPG).

In another embodiment, the preservative is selected from the group consisting of sodium sorbate, potassium sorbate, sodium benzoate and potassium benzoate or any combination thereof. In one embodiment, the liquid formulation comprises 0.02-1.5% w/w preservative, e.g., 0.05-1% w/w preservative or 0.1-0.5% w/w preservative. In one embodiment, the liquid formulation comprises 0.001-2% w/w preservative (i.e., total amount of preservative), e.g., 0.02-1.5% w/w preservative, 0.05-1% w/w preservative, or 0.1-0.5% w/w preservative, wherein the preservative is selected from the group consisting of sodium sorbate, potassium sorbate, sodium benzoate and potassium benzoate or any combination thereof.

In another embodiment, the liquid formulation further comprises one or more additional enzymes, e.g. as described above.

### Use of Hexosaminidase Variants

The hexosaminidase variants of the invention are suitable for use in a cleaning process such as laundry or hard surface cleaning, in particular for laundry. Thus, one aspect of the invention relates a method for laundering an item, wherein the item is a textile, the method comprising:
a) exposing the item to a wash liquor comprising a hexosaminidase variant of the invention;
b) completing at least one wash cycle; and optionally
c) rinsing the item.

The pH of the liquid wash liquor solution is typically in the range about 5.5 to about 10, more typically in the range of about 7 to about 9, such as in the range of about 7 to about 8.5 or about 7 to about 8.

The wash liquor may have a temperature in the range of 5° C. to 95° C., or in the range of 10° C. to 80° C., in the range of 10° C. to 70° C., in the range of 10° C. to 60° C., in the range of 10° C. to 50° C., in the range of 15° C. to 40° C. or in the range of 20° C. to 30° C.

The concentration of the hexosaminidase variant enzyme in the wash liquor is typically in the range of from 0.0001 mg/l to 10 mg/l enzyme protein, from 0.0002 mg/l to 10 mg/l, from 0.001 mg/l to 10 mg/l, from 0.002 mg/l to 10 mg/l, from 0.01 mg/l to 10 mg/l, from 0.02 mg/l to 10 mg/l, from 0.1 mg/l to 10 mg/l, from 0.2 mg/l to 10 mg/l, or from 0.2 mg/l to 5 mg/l.

In one embodiment, the hexosaminidase variant or a detergent composition comprising the variant may be used for cleaning of a hard surface, where the hard surface may for example be dishware, a surface such as a tabletop, wall or floor, or an interior surface of a machine such as a washing machine or a dishwasher.

When items like T-shirts or sportswear are used, they are exposed to bacteria from the body of the user and from the rest of the environment in which they are used. This may cause malodor on the item even after the item is washed. The present invention therefore also relates to methods for removal or reduction of malodor on textile. The malodor may be caused by bacteria that produce compounds with an unpleasant smell. One example of such unpleasant smelling compounds is E-2-nonenal. The malodor can be present on a newly washed textile which is still wet, or the malodor can be present on a newly washed textile which has subsequently been dried. The malodor may also be present on a textile which has been stored for some time after wash. The present invention thus also relates to use of a hexosaminidase variant of the invention for reduction or removal of malodor such as E-2-nonenal from wet or dry textiles.

In one embodiment, the hexosaminidase variants of the present invention have improved malodor removal properties compared to the parent or a reference hexosaminidase such as SEQ ID NO: 1, wherein the malodor is measured as described in Example 9 of WO 2017/186943.

The invention is further defined by the following numbered paragraphs:
1. A variant of the hexosaminidase polypeptide of SEQ ID NO: 1, wherein the variant comprises, compared to SEQ ID NO: 1:
   a substitution at one or more positions, preferably two, three or four positions, corresponding to positions 163, 227, 252 and 309 of SEQ ID NO: 1; and
   a substitution at one or more positions corresponding to positions 106, 111, 120, 124, 127, 150, 170, 171, 178, 199, 208, 254, 255 and 278 of SEQ ID NO: 1;
   wherein the variant has hexosaminidase activity and has at least 60%, e.g., at least 65%, at least 70%, at least 75%, at least 80%, at least 85%, at least 90% or at least 95% sequence identity, but less than 100% sequence identity, to the polypeptide of SEQ ID NO: 1, SEQ ID NO: 2 or SEQ ID NO: 3.
2. The hexosaminidase variant of paragraph 1, wherein the variant comprises, compared to SEQ ID NO: 1, two, three or four substitutions selected from the group consisting of S163P, N227T, N252P and K309E;
   and further comprises at least one substitution selected from the group consisting of V106M, V106N, D111R, T120V, Y124I, Y124K, Y124L, R127P, E150N, L170G, D171F, D171H, Q178K, S199W, S208W, F254I, T255D and I278V.
3. The hexosaminidase variant of paragraph 1 or 2, wherein the variant further comprises, compared to SEQ ID NO: 1, at least one substitution selected from the group consisting of H15Y, S186R, S225G, E232D, G235W, N260Q, H272V, S279D, Y281P, K308Q and K312Q, for example 2, 3, 4, 5, 6, 7, 8, 9, 10 or all of said substitutions.
4. The hexosaminidase variant of paragraph 3, wherein the variant further comprises, compared to SEQ ID NO: 1, one or more substitutions selected from the group consisting of Q3I, A49W and N59E, preferably 2 or all 3 of said substitutions; or one or more substitutions selected from the group consisting of Q3F, V140I, Q215K and N267T, preferably 2, 3 or all 4 of said substitutions.
5. The hexosaminidase variant of any of the preceding paragraphs, wherein the variant comprises, compared to SEQ ID NO: 1:
   the substitutions S163P, N227T, N252P and K309E; and
   at least one substitution selected from the group consisting of H15Y, S186R, S225G, E232D, G235W, N260Q, H272V, S279D, Y281P, K308Q and K312Q, for example 2, 3, 4, 5, 6, 7, 8, 9, 10 or all of said substitutions.
6. The hexosaminidase variant of any of the preceding paragraphs, wherein the variant comprises, compared to SEQ ID NO: 1, at least one substitution selected from the group consisting of V106M/N, D111R, T120V, Y124I/K/L, R127P, E150N, L170G, D171F/H, Q178K, S199W, S208W, T255D, N267T, I278V and K308E, for example at least two of said substitutions; and wherein the variant has at least 80%, at least 85%, at least 90% or at least 95% sequence identity, but less than 100% sequence identity, to SEQ ID NO: 2 or SEQ ID NO: 3.
7. The hexosaminidase variant of any of the preceding paragraphs, wherein the variant comprises, compared to SEQ ID NO: 1, at least one substitution selected from the group consisting of V106M/N, D111R, T120V, E150N, L170G, D171F, Q178K, S199W, S208W, T255D and I278V, for example at least two of said substitutions; and wherein the variant has at least 80%, at least 85%, at least 90% or at least 95% sequence identity, but less than 100% sequence identity, to SEQ ID NO: 2 or SEQ ID NO: 3.
8. The hexosaminidase variant of any of the preceding paragraphs, wherein the variant comprises, compared to SEQ ID NO: 1, at least one substitution selected from the group consisting of V106M/N, D111R, T120V, E150N, D171F and I278V, for example at least two of said substitutions; and wherein the variant has at least 80%, at least 85%, at least 90% or at least 95% sequence identity, but less than 100% sequence identity, to SEQ ID NO: 2 or SEQ ID NO: 3.
9. The hexosaminidase variant of any of the preceding paragraphs, wherein the variant comprises at least one substitution selected from the group consisting of D111R, T120V and E150N; and wherein the variant has at least 80%, at least 85%, at least 90% or at least 95% sequence identity, but less than 100% sequence identity, to SEQ ID NO: 2 or SEQ ID NO: 3.
10. The hexosaminidase variant of paragraph 9, wherein the variant comprises the substitutions D111R+T120V, D111R+E150N, T120V+E150N or all three substitutions D111R+T120V+E150N.
11. The hexosaminidase variant of any of paragraphs 1-10, wherein the variant comprises, compared to SEQ ID NO: 2, at least one of the following substitutions or sets of substitutions:
   D111R
   D111R+T120V
   D111R+T120V+E150N
   D111R+T120V+E150N+D171F+I278V
   D111R+T120V+T255D
   D111R+T120V+Y124I
   D111R+T120V+Y124K+Q178K
   D111R+T120V+Y124L+Q178K
   D111R+Y124K+D171FD171F+S208W+N267T
   D171F+T255D
   D171H
   I278V
   K308E
   N267T
   R127P
   T120V+D171F
   T120V+D171F+N267T
   T120V+S208W+N267T
   T120V+T255D
   T120V+T255D+N267T
   T120V+Y124K+Q178K
   V106M+D111R+T120V+D171F
   V106M+Q178K+T255D
   V106M+T120V+D171F+Q178K
   V106M+T120V+Y124I+D171F
   V106M+T120V+Y124I+Q178K
   V106M+T120V+Y124K+D171F
   V106M+T120V+Y124L+Q178K
   V106N+D111R+T120V+E150N
   V106N+S208W+N267T
   V106N+T120V+D171F
   V106N+T120V+D171F+S208W
   V106N+T120V+E150N+D171F+I278V
   V106N+T120V+N267T
12. The hexosaminidase variant of any of paragraphs 1-10, wherein the variant comprises, compared to SEQ ID NO: 3, at least one of the following substitutions or sets of substitutions:
   L170G
   E150N+F254I
   F254I
   V106N
   D111R+T120V
   D111R+T120V+E150N
   D111R+T120V+E150N+D171F+I278V
   D111R+T120V+T255D
   D111R+T120V+Y124I
   D111R+T120V+Y124K+Q178K
   D111R+T120V+Y124L+Q178K
   D111R+Y124K+D171F
   D171F+S208W+N267T
   D171F+T255D
   D171H
   I278V
   K308E
   N267T
   R127P
   T120V+D171F
   T120V+D171F+N267T
   T120V+S208W+N267T
   T120V+T255D
   T120V+T255D+N267T
   T120V+Y124K+Q178K
   V106M+D111R+T120V+D171F
   V106M+Q178K+T255D
   V106M+T120V+D171F+Q178K
   V106M+T120V+Y124I+D171F
   V106M+T120V+Y124I+Q178K
   V106M+T120V+Y124K+D171F
   V106M+T120V+Y124L+Q178K
   V106N+D111R+T120V+E150N
   V106N+S208W+N267T
   V106N+T120V+D171F
   V106N+T120V+D171F+S208W
   V106N+T120V+E150N+D171F+I278V
   V106N+T120V+N267T
13. The hexosaminidase variant of any of the preceding paragraphs, wherein the variant comprises one of the following sets of substitutions compared to SEQ ID NO: 1:
   Q3I+H15Y+A49W+N59E+D111R+T120V+S163P+S186R+S225G+N227T+E232D+G235W+N252P+N260Q+H272V+S279D+Y281P+K308Q+K309E+K312Q
   Q3I+H15Y+A49W+N59E+D111R+T120V+E150N+S163P+S186R+S225G+N227T+E232D+G235W+N252P+N260Q+H272V+S279D+Y281P+K308Q+K309E+K312Q
   Q3I+H15Y+A49W+N59E+D111R+T120V+E150N+S163P+D171F+S186R+S225G+N227T+E232D+G235W+N252P+N260Q+H272V+I278V+S279D+Y281P+K308Q+K309E+K312Q
   Q3I+H15Y+A49W+N59E+D111R+T120V+S163P+S186R+S225G+N227T+E232D+G235W+N252P+T255D+N260Q+H272V+S279D+Y281P+K308Q+K309E+K312Q
   Q3I+H15Y+A49W+N59E+D111R+T120V+Y124I+S163P+S186R+S225G+N227T+E232D+G235W+N252P+N260Q+H272V+S279D+Y281P+K308Q+K309E+K312Q
   Q3I+H15Y+A49W+N59E+D111R+T120V+Y124K+S163P+Q178K+S186R+S225G+N227T+E232D+G235W+N252P+N260Q+H272V+S279D+Y281P+K308Q+K309E+K312Q
   Q3I+H15Y+A49W+N59E+D111R+T120V+Y124L+S163P+Q178K+S186R+S225G+N227T+E232D+G235W+N252P+N260Q+H272V+S279D+Y281P+K308Q+K309E+K312Q
   Q3I+H15Y+A49W+N59E+D111R+Y124K+S163P+D171F+S186R+S225G+N227T+E232D+G235W+N252P+N260Q+H272V+S279D+Y281P+K308Q+K309E+K312Q
   Q3I+H15Y+A49W+N59E+S163P+D171F+S186R+S208W+S225G+N227T+E232D+G235W+N252P+N260Q+N267T+H272V+S279D+Y281P+K308Q+K309E+K312Q
   Q3I+H15Y+A49W+N59E+S163P+D171F+S186R+S225G+N227T+E232D+G235W+N252P+T255D+N260Q+H272V+S279D+Y281P+K308Q+K309E+K312Q
   Q3I+H15Y+A49W+N59E+S163P+D171H+S186R+S225G+N227T+E232D+G235W+N252P+N260Q+H272V+S279D+Y281P+K308Q+K309E+K312Q
   Q3I+H15Y+A49W+N59E+S163P+S186R+S225G+N227T+E232D+G235W+N252P+N260Q+H272V+I278V+S279D+Y281P+K308Q+K309E+K312Q
   Q3I+H15Y+A49W+N59E+S163P+S186R+S225G+N227T+E232D+G235W+N252P+N260Q+H272V+S279D+Y281P+K308E+K309E+K312Q
   Q3I+H15Y+A49W+N59E+S163P+S186R+S225G+N227T+E232D+G235W+N252P+N260Q+N267T+H272V+S279D+Y281P+K308Q+K309E+K312Q
   Q3I+H15Y+A49W+N59E+R127P+S163P+S186R+S225G+N227T+E232D+G235W+N252P+N260Q+H272V+S279D+Y281P+K308Q+K309E+K312Q
   Q3I+H15Y+A49W+N59E+T120V+S163P+D171F+S186R+S225G+N227T+E232D+G235W+N252P+N260Q+H272V+S279D+Y281P+K308Q+K309E+K312Q
   Q3I+H15Y+A49W+N59E+T120V+S163P+D171F+S186R+S225G+N227T+E232D+G235W+N252P+N260Q+N267T+H272V+S279D+Y281P+K308Q+K309E+K312Q
   Q3I+H15Y+A49W+N59E+T120V+S163P+S186R+S208W+S225G+N227T+E232D+G235W+N252P+N260Q+N267T+H272V+S279D+Y281P+K308Q+K309E+K312Q
   Q3I+H15Y+A49W+N59E+T120V+S163P+S186R+S225G+N227T+E232D+G235W+N252P+T255D+N260Q+H272V+S279D+Y281P+K308Q+K309E+K312Q
   Q3I+H15Y+A49W+N59E+T120V+S163P+S186R+S225G+N227T+E232D+G235W+N252P+T255D+N260Q+N267T+H272V+S279D+Y281P+K308Q+K309E+K312Q
   Q3I+H15Y+A49W+N59E+T120V+Y124K+S163P+Q178K+S186R+S225G+N227T+E232D+G235W+N252P+N260Q+H272V+S279D+Y281P+K308Q+K309E+K312Q
   Q3I+H15Y+A49W+N59E+V106M+D111R+T120V+S163P+D171F+S186R+S225G+N227T+E232D+G235W+N252P+N260Q+H272V+S279D+Y281P+K308Q+K309E+K312Q
   Q3I+H15Y+A49W+N59E+V106M+S163P+Q178K+S186R+S225G+N227T+E232D+G235W+N252P+T255D+N260Q+H272V+S279D+Y281P+K308Q+K309E+K312Q
   Q3I+H15Y+A49W+N59E+V106M+T120V+S163P+D171F+Q178K+S186R+S225G+N227T+E232D+G235W+N252P+N260Q+H272V+S279D+Y281P+K308Q+K309E+K312Q
   Q3I+H15Y+A49W+N59E+V106M+T120V+Y124I+S163P+D171F+S186R+S225G+N227T+E232D+G235W+N252P+N260Q+H272V+S279D+Y281P+K308Q+K309E+K312Q
   Q3I+H15Y+A49W+N59E+V106M+T120V+Y124I+S163P+Q178K+S186R+S225G+N227T+E232D+G235W+N252P+N260Q+H272V+S279D+Y281P+K308Q+K309E+K312Q
   Q3I+H15Y+A49W+N59E+V106M+T120V+Y124K+S163P+D171F+S186R+S225G+N227T+E232D+G235W+N252P+N260Q+H272V+S279D+Y281P+K308Q+K309E+K312Q
   Q3I+H15Y+A49W+N59E+V106M+T120V+Y124L+S163P+Q178K+S186R+S225G+N227T+E232D+G235W+N252P+N260Q+H272V+S279D+Y281P+K308Q+K309E+K312Q
   Q3I+H15Y+A49W+N59E+V106N+D111R+T120V+E150N+S163P+S186R+S225G+N227T+E232D+G235W+N252P+N260Q+H272V+S279D+Y281P+K308Q+K309E+K312Q
   Q3I+H15Y+A49W+N59E+V106N+S163P+S186R+S208W+S225G+N227T+E232D+G235W+N252P+N260Q+N267T+H272V+S279D+Y281P+K308Q+K309E+K312Q
   Q3I+H15Y+A49W+N59E+V106N+T120V+S163P+D171F+S186R+S225G+N227T+E232D+G235W+N252P+N260Q+H272V+S279D+Y281P+K308Q+K309E+K312Q
   Q3I+H15Y+A49W+N59E+V106N+T120V+S163P+D171F+S186R+S208W+S225G+N227T+E232D+G235W+N252P+N260Q+H272V+S279D+Y281P+K308Q+K309E+K312Q
   Q3I+H15Y+A49W+N59E+V106N+T120V+E150N+S163P+D171F+S186R+S225G+N227T+E232D+G235W+N252P+N260Q+H272V+I278V+S279D+Y281P+K308Q+K309E+K312Q
   Q3I+H15Y+A49W+N59E+V106N+T120V+S163P+S186R+S225G+N227T+E232D+G235W+N252P+N260Q+N267T+H272V+S279D+Y281P+K308Q+K309E+K312Q
   Q3F+H15Y+V140I+S163P+L170G+S186R+Q215K+S225G+N227T+E232D+G235W+N252P+N260Q+N267T+H272V+S279D+Y281P+K308Q+K309E+K312Q
   Q3F+H15Y+V140I+E150N+S163P+S186R+Q215K+S225G+N227T+E232D+G235W+N252P+F254I+N260Q+N267T+H272V+S279D+Y281P+K308Q+K309E+K312Q
Q3F+H15Y+V140I+S163P+S186R+Q215K+S225G+N227T+E232D+G235W+N252P+F254I+N260Q+N267T+H272V+S279D+Y281P+K308Q+K309E+K312Q
   Q3F+H15Y+V106N+V140I+S163P+S186R+Q215K+S225G+N227T+E232D+G235W+N252P+N260Q+N267T+H272V+S279D+Y281P+K308Q+K309E+K312Q
   Q3F+H15Y+D111R+V140I+S163P+S186R+Q215K+S225G+N227T+E232D+G235W+N252P+N260Q+N267T+H272V+S279D+Y281P+K308Q+K309E+K312Q
   Q3F+H15Y+D111R+V140I+E150N+S163P+S186R+Q215K+S225G+N227T+E232D+G235W+N252P+N260Q+N267T+H272V+S279D+Y281P+K308Q+K309E+K312Q
   Q3F+H15Y+D111R+V140I+E150N+S163P+L170G+S186R+Q215K+S225G+N227T+E232D+G235W+N252P+N260Q+N267T+H272V+S279D+Y281P+K308Q+K309E+K312Q
   Q3F+H15Y+D111R+V140I+E150N+S163P+L170G+S186R+Q215K+S225G+N227T+E232D+G235W+N252P+N260Q+N267T+H272V+I278V+S279D+Y281P+K308Q+K309E+K312Q
   Q3F+H15Y+D111R+V140I+E150N+S163P+S186R+S199W+Q215K+S225G+N227T+E232D+G235W+N252P+N260Q+N267T+H272V+I278V+S279D+Y281P+K308Q+K309E+K312Q
   Q3F+H15Y+D111R+V140I+S163P+S186R+Q215K+S225G+N227T+E232D+G235W+N252P+N260Q+N267T+H272V+I278V+S279D+Y281P+K308Q+K309E+K312Q
   Q3F+H15Y+D111R+V140I+S163P+L170G+S186R+Q215K+S225G+N227T+E232D+G235W+N252P+N260Q+N267T+H272V+S279D+Y281P+K308Q+K309E+K312Q
   Q3F+H15Y+D111R+V140I+S163P+L170G+S186R+Q215K+S225G+N227T+E232D+G235W+N252P+N260Q+N267T+H272V+I278V+S279D+Y281P+K308Q+K309E+K312Q
   Q3F+H15Y+D111R+V140I+S163P+L170G+S186R+S199W+Q215K+S225G+N227T+E232D+G235W+N252P+N260Q+N267T+H272V+S279D+Y281P+K308Q+K309E+K312Q
   Q3F+H15Y+V140I+E150N+S163P+S186R+Q215K+S225G+N227T+E232D+G235W+N252P+N260Q+N267T+H272V+S279D+Y281P+K308Q+K309E+K312Q
   Q3F+H15Y+V140I+E150N+S163P+S186R+Q215K+S225G+N227T+E232D+G235W+N252P+N260Q+N267T+H272V+I278V+S279D+Y281P+K308Q+K309E+K312Q
   Q3F+H15Y+V140I+E150N+S163P+L170G+S186R+Q215K+S225G+N227T+E232D+G235W+N252P+N260Q+N267T+H272V+S279D+Y281P+K308Q+K309E+K312Q
   Q3F+H15Y+V140I+E150N+S163P+L170G+S186R+Q215K+S225G+N227T+E232D+G235W+N252P+N260Q+N267T+H272V+I278V+S279D+Y281P+K308Q+K309E+K312Q
   Q3F+H15Y+V140I+E150N+S163P+S186R+S199W+Q215K+S225G+N227T+E232D+G235W+N252P+N260Q+N267T+H272V+S279D+Y281P+K308Q+K309E+K312Q
   Q3F+H15Y+V140I+S163P+S186R+Q215K+S225G+N227T+E232D+G235W+N252P+N260Q+N267T+H272V+I278V+S279D+Y281P+K308Q+K309E+K312Q
   Q3F+H15Y+V140I+S163P+L170G+S186R+Q215K+S225G+N227T+E232D+G235W+N252P+N260Q+N267T+H272V+S279D+Y281P+K308Q+K309E+K312Q
   Q3F+H15Y+V140I+S163P+L170G+S186R+Q215K+S225G+N227T+E232D+G235W+N252P+N260Q+N267T+H272V+I278V+S279D+Y281P+K308Q+K309E+K312Q
   Q3F+H15Y+V140I+S163P+L170G+S186R+S199W+Q215K+S225G+N227T+E232D+G235W+N252P+N260Q+N267T+H272V+S279D+Y281P+K308Q+K309E+K312Q
   Q3F+H15Y+V140I+S163P+L170G+S186R+S199W+Q215K+S225G+N227T+E232D+G235W+N252P+N260Q+N267T+H272V+I278V+S279D+Y281P+K308Q+K309E+K312Q
   Q3F+H15Y+V140I+S163P+S186R+S199W+Q215K+S225G+N227T+E232D+G235W+N252P+N260Q+N267T+H272V+I278V+S279D+Y281P+K308Q+K309E+K312Q
14. The hexosaminidase variant of any of the preceding paragraphs, wherein the variant has at least 80%, at least 85%, at least 90% or at least 95% sequence identity, but less than 100% sequence identity, to SEQ ID NO: 2 or SEQ ID NO: 3.
15. The hexosaminidase variant of any of the preceding paragraphs, wherein the variant has improved stability compared to the polypeptide of SEQ ID NO: 1, SEQ ID NO: 2 or SEQ ID NO: 3, or to a parent polypeptide having the same amino acid sequence as the variant without said substitutions.
16. The hexosaminidase variant of paragraph 15, wherein the improved stability is selected from the group consisting of detergent stability and thermostability.
17. A detergent composition comprising the hexosaminidase variant of any of paragraphs 1-16 and at least one detergent adjunct ingredient.
18. Use of the hexosaminidase variant of any of paragraphs 1-16 or the detergent composition of paragraph 17 in a cleaning process, such as laundry or hard surface cleaning such as dishwashing.
19. A method of cleaning an item, comprising exposing the item to a wash liquor comprising the hexosaminidase variant of any of paragraphs 1-16 or to the detergent composition of paragraph 17.
20. The method of paragraph 19, wherein the item is a textile or a hard surface, for example dishware or a surface such as a tabletop, wall or floor or an interior surface of a machine such as a washing machine or a dishwasher.
21. A method for laundering a textile, comprising:
   a) exposing the textile to a wash liquor comprising the hexosaminidase variant of any of paragraphs 1-16 or the detergent composition of paragraph 17;
   b) completing at least one wash cycle; and optionally
   c) rinsing the item.
22. A granule comprising:
   (a) a core comprising the hexosaminidase variant of any one of paragraphs 1-17, and, optionally
   (b) a coating consisting of one or more layers surrounding the core.
23. A liquid composition comprising the hexosaminidase variant of any one of paragraphs 1-17 and an enzyme stabilizer, e.g., a polyol such as propylene glycol or glycerol, a sugar or sugar alcohol, lactic acid, a reversible protease inhibitor, boric acid, or a boric acid derivative, e.g., an aromatic borate ester, or a phenyl boronic acid derivative such as 4-formylphenyl boronic acid.
24. A polynucleotide encoding the hexosaminidase variant of any of paragraphs 1-17, a nucleic acid construct or expression vector comprising the polynucleotide, or a recombinant host cell transformed with the polynucleotide.
25. A method of producing a hexosaminidase variant, comprising:
   a) cultivating the recombinant host cell of paragraph 24 under conditions suitable for expression of the variant; and
   b) recovering the variant.

The present invention is further described by the following examples that should not be construed as limiting the scope of the invention.

### EXAMPLES

### Materials and Methods

### Hexosaminidase Activity Assays

Hexosaminidase activity may be e.g. be determined using one of the following assays.

### Assay 1

The hexosaminidase activity of hexosaminidase polypeptides may be determined using 4-nitrophenyl N-acetyl-β-D-glucosaminide (Sigma-Aldrich) as substrate. The enzymatic reaction is performed in triplicate in a 96 well flat bottom polystyrene microtiter plate (Thermo Scientific) with the following conditions: 50 mM 2-(N-morpholino) ethanesulfonic acid pH 6 buffer, 1.5 mg/ml 4-nitrophenyl N-acetyl-β-D-glucosaminide and 20 μg/ml purified enzyme sample in a total reaction volume of 100 μl. Blank samples without enzyme are run in parallel. The reactions are carried out at 37° C. in a Thermomixer comfort (Eppendorf). After 10 minutes of incubation, 5 μl 1 M NaOH is added to each reaction mixture to stop the enzymatic reaction. The absorbance is read at 405 nm using a POLARstar Omega plate reader (BMG LABTECH) to estimate the formation of 4-nitrophenolate ion released due to enzymatic hydrolysis of the 4-nitrophenyl N-acetyl-β-D-glucosaminide substrate. An increased absorbance over the blank (difference in absorbance at 405 nm between the sample and the blank) indicates hexosaminidase activity.

### Assay 2

The hexosaminidase activity of hexosaminidase polypeptides may be determined using 4-methylumbeliferyl N-acetyl-β-D-glucosaminide (Sigma-Aldrich) as substrate. The enzymatic reaction is performed in triplicate in a 96 well flat bottom polystyrene microtiter plate (Thermo Scientific) with the following conditions: 20 mM 3-morpholinopropane-1-sulfonic acid pH 7 buffer, 5 mM 4-methylumbeliferyl N-acetyl-β-D-glucosaminide and 20 nM purified enzyme sample in a total reaction volume of 200 μl. Blank samples without enzyme are run in parallel. The reactions are carried out at ambient temperature 20-25° C. The reaction kinetics are followed immediately after mixing of enzyme and substrate using a SpectraMax M2e plate reader. Excitation wavelength is set to 368 nm and fluorescence emission reading is done at 448 nm. The reaction is followed for 30 min at 60 second intervals. The increase in fluorescence signal is used to estimate the formation of 4-methylumbeliferyl ion released due to enzymatic hydrolysis of the 4-methylumbeliferyl N-acetyl-β-D-glucosaminide substrate.

The results are expressed as an average initial rate of reaction measured as relative fluorescence units per minute (RFU/min) using excitation at 368 nm and fluorescence emission at 448 nm for each reaction performed in triplicate. An increased value over the blank (A RFU/min) indicates hexosaminidase activity.

### Growth and Expression

Hexosaminidase polypeptides were expressed in Bacillus host cells grown in standard 96-well microtiter plates (200 μl broth/well). The broth used for growth and expression was Cal18-2 (Ostergaard et al., 2010, Identification and characterization of a bacterial glutamic peptidase, BMC Biochem. 11:47) supplemented with 6 μg/ml chloramphenicol. The microtiter plates were grown for 3 days at 30° C. with shaking at 225 rpm. After growth, the plates were centrifuged, and the supernatants were stressed as described below and assayed for stability. All steps related to growth, stress and assaying were done in 96- or 384-well format microtiter plates.

### Measurement of Stability of Variants in Supernatant in Liquid Detergent

Supernatants were diluted in concentrated 80% (v/v) Persil® ProClean® Power Liquid 2in1 detergent (Henkel). After mixing, the samples were split in two parts. One part was incubated at room temperature (21° C., unstressed sample) for the chosen stress time, and the second part was incubated in a PCR machine at the chosen elevated stress temperature and for the chosen stress time (“stressed sample”). The applied stress conditions can be found in the individual examples below. After incubation, the samples were diluted 8-fold in dilution buffer (100 mM Tris-HCl, 0.01% (v/v) TritonX-100, pH 8.0) before the activity of the unstressed and stressed samples was determined by transferring 5 μl sample to a 384-well microtiter plate containing 35 μl assay solution (45 mM citrate buffer pH 5.0 supplemented with 1.0 mg/ml p-nitrophenyl-N-acetyl-β-D-glucosaminide (Sigma Aldrich). After incubation at room temperature for 9 hours, the reactions were stopped by adding 40 μl stop solution (0.4 M Na₂CO₃) and the absorbance at 405 nm (A405) was read. For each sample the residual activity (RA) was calculated as:

RA=A405(stressed sample)/A405(unstressed sample).

All A405 measurements were corrected for the signal of a blank sample (no hexosaminidase present) before calculating the RA value. The RA values were used to calculate the half-life of the variants and the backbone (SEQ ID NO: 2, SEQ ID NO: 3, or a reference hexosaminidase as indicated in the individual examples): Half-life (minutes)=60 (minutes)×Ln(0.5)/Ln(RA) of each variant and the backbone. For each variant the half-life improvement factor (HIF) is calculated as: HIF=Half-life(variant)/Half-life(backbone). Variants with improved stability will have a HIF>1, since the HIF of the backbone in this setup will by definition be 1.

### Purified Variants

### Growth, Expression, Purification

For purification, hexosaminidase polypeptides were expressed in Bacillus host cells and grown in 500 ml shake flasks each containing 100 ml PS-1 media. The shake flasks were grown for 4 days at 30° C. with shaking at 270 rpm. The culture broth was centrifuged (26,000×g, 20 min) and the supernatant was carefully decanted from the precipitate. The supernatant was filtered through a Nalgene 0.2 μm filtration unit to remove the rest of the Bacillus host cells. The 0.2 μm filtrate was transferred to 20 mM MES/NaOH, pH 6.0 on a G25 Sephadex column (GE Healthcare). The G25 transferred solution was applied to a SOURCE Q column (GE Healthcare) equilibrated in 20 mM MES/NaOH, pH 6.0. After washing the column extensively with the equilibration buffer, the hexosaminidase was eluted with a linear NaCl gradient (0→1.0 M NaCl) in the same buffer over five column volumes. Fractions were collected during elution and the collected fractions were analysed by SDS-PAGE. Fractions in which only one band was seen after Coomassie staining were pooled as the purified preparation and used for further experiments.

### Determination of Stability (Half-Life) of Purified Variants

The stability, expressed as half-life, of mature polypeptides purified as described above was determined by incubation of 300 μL, 5 μM of each variant sample in 96-well flat-bottom polystyrene microtiter plates (Thermo Scientific) at 37° C. for 7 days under the following conditions: 20 vol % EPPS (100 mM, pH 8.3), 0.01 wt % Triton X-100 and 80 vol % Persil® ProClean® Power Liquid 2in1 detergent (=“stress plate”). On days 1, 3, and 7, 20 μL aliqouts from the stress plate were analysed for residual activity using 4-methylumbeliferyl N-acetyl-D-glucosaminide (Sigma-Aldrich) as substrate: a 20 μL sample from the stress plate and 100 μL substrate in citrate buffer (45 mM, pH 5.3, 0.01 wt % Brij®L23) was mixed to concentrations of 208 nM sample in solution with 6 mM substrate. The reaction mixture was heated to 45° C. for 90 min in a PCR incubator and stopped by addition of 120 μL Na₂CO₃ (0.6 M, pH 10.3). Absorbance was measured at 405 nm in a standard plate spectrophotometer (SpectraMax 5e).

The half-life of the stressed samples was estimated by linearization of sample activity decay data (log(Activity) vs time), followed by linear regression to provide a determination of the samples' half-life (t ½). A half-life improvement factor (HIF) was calculated as the ratio between the half-life of the hexosaminidase variant and the half-life of the reference hexosaminidase:

HIF=t½(variant)/t½(reference)

### Example 1: Stability of Hexosaminidase Variants

The detergent stability of hexosaminidase variants of the invention in supernatant was determined as a half-life compared to the half-life of the hexosaminidase of SEQ ID NO: 3 using the methods described above. The stress conditions in this example were 62° C. for 210 min in 80% Persil® ProClean® Power Liquid 2in1 detergent. The results are expressed as a half-life improvement factor, HIF, compared to the half-life of SEQ ID NO: 3, in Table 1 below.

TABLE 1
Half-life improvement factor, HIF, of hexosaminidase variants

| | Substitutions | HIF compared |
|---|---|---|
| | compared to | to SEQ ID |
| Substitutions compared to SEQ ID NO: 1 | SEQ ID NO: 3 | NO: 3 |
| Q3F H15Y V140I S163P L170G S186R Q215K S225G | L170G | 2.87 |
| N227T E232D G235W N252P N260Q N267T H272V | | |
| S279D Y281P K308Q K309E K312Q | | |
| Q3F H15Y V140I E150N S163P S186R Q215K S225G | E150N F254I | 1.69 |
| N227T E232D G235W N252P F254I N260Q N267T | | |
| H272V S279D Y281P K308Q K309E K312Q | | |
| Q3F H15Y V140I S163P S186R Q215K S225G N227T | F254I | 2.64 |
| E232D G235W N252P F254I N260Q N267T H272V | | |
| S279D Y281P K308Q K309E K312Q | | |
| Q3F H15Y V106N V140I S163P S186R Q215K S225G | V106N | 3.42 |
| N227T E232D G235W N252P N260Q N267T H272V | | |
| S279D Y281P K308Q K309E K312Q | | |

### Example 2: Stability of Hexosaminidase Variants

The detergent stability of hexosaminidase variants of the invention in supernatant was determined as a half-life compared to the half-life of the hexosaminidase of SEQ ID NO: 2 using the methods described above, with the stress conditions being a temperature of 57° C. and an incubation time of 360 min. The results are expressed as a half-life improvement factor, HIF, compared to the half-life of the hexosaminidase of SEQ ID NO: 2, in Table 2 below.

TABLE 2
Half-life improvement factor, HIF, of hexosaminidase variants

| | Substitutions | HIF compared |
|---|---|---|
| | compared to | to SEQ ID |
| Substitutions compared to SEQ ID NO: 1 | SEQ ID NO: 2 | NO: 2 |
| Q3I H15Y A49W N59E D111R T120V S163P S186R | D111R T120V | 1.59 |
| S225G N227T E232D G235W N252P N260Q H272V | | |
| S279D Y281P K308Q K309E K312Q | | |
| Q3I H15Y A49W N59E D111R T120V E150N S163P | D111R T120V | 1.52 |
| S186R S225G N227T E232D G235W N252P N260Q | E150N | |
| H272V S279D Y281P K308Q K309E K312Q | | |
| Q3I H15Y A49W N59E D111R T120V E150N S163P | D111R T120V | 2.41 |
| D171F S186R S225G N227T E232D G235W N252P | E150N D171F | |
| N260Q H272V I278V S279D Y281P K308Q K309E | I278V | |
| K312Q | | |
| Q3I H15Y A49W N59E D111R T120V S163P S186R | D111R T120V | 2.36 |
| S225G N227T E232D G235W N252P T255D N260Q | T255D | |
| H272V S279D Y281P K308Q K309E K312Q | | |
| Q3I H15Y A49W N59E D111R T120V Y124I S163P | D111R T120V | 2.83 |
| S186R S225G N227T E232D G235W N252P N260Q | Y124I | |
| H272V S279D Y281P K308Q K309E K312Q | | |
| Q3I H15Y A49W N59E D111R T120V Y124K S163P | D111R T120V | 1.74 |
| Q178K S186R S225G N227T E232D G235W N252P | Y124K Q178K | |
| N260Q H272V S279D Y281P K308Q K309E K312Q | | |
| Q3I H15Y A49W N59E D111R T120V Y124L S163P | D111R T120V | 2.04 |
| Q178K S186R S225G N227T E232D G235W N252P | Y124L Q178K | |
| N260Q H272V S279D Y281P K308Q K309E K312Q | | |
| Q3I H15Y A49W N59E D111R Y124K S163P D171F | D111R Y124K | 2.07 |
| S186R S225G N227T E232D G235W N252P N260Q | D171F | |
| H272V S279D Y281P K308Q K309E K312Q | | |
| Q3I H15Y A49W N59E S163P D171F S186R S208W | D171F S208W | 3.25 |
| S225G N227T E232D G235W N252P N260Q N267T | N267T | |
| H272V S279D Y281P K308Q K309E K312Q | | |
| Q3I H15Y A49W N59E S163P D171F S186R S225G | D171F T255D | 1.63 |
| N227T E232D G235W N252P T255D N260Q H272V | | |
| S279D Y281P K308Q K309E K312Q | | |
| Q3I H15Y A49W N59E S163P D171H S186R S225G | D171H | 2.81 |
| N227T E232D G235W N252P N260Q H272V S279D | | |
| Y281P K308Q K309E K312Q | | |
| Q3I H15Y A49W N59E S163P S186R S225G N227T | I278V | 1.94 |
| E232D G235W N252P N260Q H272V I278V S279D | | |
| Y281P K308Q K309E K312Q | | |
| Q3I H15Y A49W N59E S163P S186R S225G N227T | K308E | 1.75 |
| E232D G235W N252P N260Q H272V S279D Y281P | | |
| K308E K309E K312Q | | |
| Q3I H15Y A49W N59E S163P S186R S225G N227T | N267T | 3.28 |
| E232D G235W N252P N260Q N267T H272V S279D | | |
| Y281P K308Q K309E K312Q | | |
| Q3I H15Y A49W N59E R127P S163P S186R S225G | R127P | 1.86 |
| N227T E232D G235W N252P N260Q H272V S279D | | |
| Y281P K308Q K309E K312Q | | |
| Q3I H15Y A49W N59E T120V S163P D171F S186R | T120V D171F | 1.61 |
| S225G N227T E232D G235W N252P N260Q H272V | | |
| S279D Y281P K308Q K309E K312Q | | |
| Q3I H15Y A49W N59E T120V S163P D171F S186R | T120V D171F | 2.88 |
| S225G N227T E232D G235W N252P N260Q N267T | N267T | |
| H272V S279D Y281P K308Q K309E K312Q | | |
| Q3I H15Y A49W N59E T120V S163P S186R S208W | T120V S208W | 2.70 |
| S225G N227T E232D G235W N252P N260Q N267T | N267T | |
| H272V S279D Y281P K308Q K309E K312Q | | |
| Q3I H15Y A49W N59E T120V S163P S186R S225G | T120V T255D | 2.00 |
| N227T E232D G235W N252P T255D N260Q H272V | | |
| S279D Y281P K308Q K309E K312Q | | |
| Q3I H15Y A49W N59E T120V S163P S186R S225G | T120V T255D | 2.04 |
| N227T E232D G235W N252P T255D N260Q N267T | N267T | |
| H272V S279D Y281P K308Q K309E K312Q | | |
| Q3I H15Y A49W N59E T120V Y124K S163P Q178K | T120V Y124K | 1.54 |
| S186R S225G N227T E232D G235W N252P N260Q | Q178K | |
| H272V S279D Y281P K308Q K309E K312Q | | |
| Q3I H15Y A49W N59E V106M D111R T120V S163P | V106M D111R | 2.03 |
| D171F S186R S225G N227T E232D G235W N252P | T120V D171F | |
| N260Q H272V S279D Y281P K308Q K309E K312Q | | |
| Q3I H15Y A49W N59E V106M S163P Q178K S186R | V106M Q178K | 2.14 |
| S225G N227T E232D G235W N252P T255D N260Q | T255D | |
| H272V S279D Y281P K308Q K309E K312Q | | |
| Q3I H15Y A49W N59E V106M T120V S163P D171F | V106M T120V | 1.73 |
| Q178K S186R S225G N227T E232D G235W N252P | D171F Q178K | |
| N260Q H272V S279D Y281P K308Q K309E K312Q | | |
| Q3I H15Y A49W N59E V106M T120V Y124I S163P | V106M T120V | 2.20 |
| D171F S186R S225G N227T E232D G235W N252P | Y124I D171F | |
| N260Q H272V S279D Y281P K308Q K309E K312Q | | |
| Q3I H15Y A49W N59E V106M T120V Y124I S163P | V106M T120V | 2.06 |
| Q178K S186R S225G N227T E232D G235W N252P | Y124I Q178K | |
| N260Q H272V S279D Y281P K308Q K309E K312Q | | |
| Q3I H15Y A49W N59E V106M T120V Y124K S163P | V106M T120V | 2.85 |
| D171F S186R S225G N227T E232D G235W N252P | Y124K D171F | |
| N260Q H272V S279D Y281P K308Q K309E K312Q | | |
| Q3I H15Y A49W N59E V106M T120V Y124L S163P | V106M T120V | 2.32 |
| Q178K S186R S225G N227T E232D G235W N252P | Y124L Q178K | |
| N260Q H272V S279D Y281P K308Q K309E K312Q | | |
| Q3I H15Y A49W N59E V106N D111R T120V E150N | V106N D111R | 2.09 |
| S163P S186R S225G N227T E232D G235W N252P | T120V E150N | |
| N260Q H272V S279D Y281P K308Q K309E K312Q | | |
| Q3I H15Y A49W N59E V106N S163P S186R S208W | V106N S208W | 1.84 |
| S225G N227T E232D G235W N252P N260Q N267T | N267T | |
| H272V S279D Y281P K308Q K309E K312Q | | |
| Q3I H15Y A49W N59E V106N T120V S163P D171F | V106N T120V | 1.53 |
| S186R S225G N227T E232D G235W N252P N260Q | D171F | |
| H272V S279D Y281P K308Q K309E K312Q | | |
| Q3I H15Y A49W N59E V106N T120V S163P D171F | V106N T120V | 1.67 |
| S186R S208W S225G N227T E232D G235W N252P | D171F S208W | |
| N260Q H272V S279D Y281P K308Q K309E K312Q | | |
| Q3I H15Y A49W N59E V106N T120V E150N S163P | V106N T120V | 1.52 |
| D171F S186R S225G N227T E232D G235W N252P | E150N D171F | |
| N260Q H272V I278V S279D Y281P K308Q K309E | I278V | |
| K312Q | | |
| Q3I H15Y A49W N59E V106N T120V S163P S186R | V106N T120V | 3.75 |
| S225G N227T E232D G235W N252P N260Q N267T | N267T | |
| H272V S279D Y281P K308Q K309E K312Q | | |

### Example 3: Stability of Hexosaminidase Variants

The detergent stability of hexosaminidase variants of the invention in supernatant was determined as a half-life compared to the half-life of SEQ ID NO: 4, which is a variant of SEQ ID NO: 1 with the substitutions S163P+Q215K+N227T+N252P+F276A+K308E+K309E+K312E, using the methods described above. The stress conditions were a temperature of 62° C. and an incubation time of 150 min. The variants tested in this example were variants of SEQ ID NO: 3 with one or more substitutions as indicated in Table 3 below, and the results are expressed as a half-life improvement factor, HIF, compared to the half-life of the reference hexosaminidase of SEQ ID NO: 4.

TABLE 3
Half-life improvement factor, HIF, of hexosaminidase variants

| Substitutions compared to | HIF compared to reference hexos- |
|---|---|
| SEQ ID NO: 3 | aminidase (SEQ ID NO: 4) |
| SEQ ID NO: 3 | 1.30 |
| D111R | 1.84 |
| D111R E150N | 2.10 |
| D111R E150N L170G | 2.54 |
| D111R E150N L170G I278V | 1.71 |
| D111R E150N S199W I278V | 1.69 |
| D111R I278V | 3.73 |
| D111R L170G | 1.88 |
| D111R L170G I278V | 3.45 |
| D111R L170G S199W | 2.37 |
| E150N | 3.50 |
| E150N I278V | 3.42 |
| E150N L170G | 2.46 |
| E150N L170G I278V | 1.82 |
| E150N S199W | 1.73 |
| I278V | 3.08 |
| L170G | 2.65 |
| L170G I278V | 1.62 |
| L170G S199W | 1.53 |
| L170G S199W I278V | 2.17 |
| S199W I278V | 1.53 |

### Example 4: Stability of Purified Hexosaminidase Variants

The detergent stability of hexosaminidase variants of the invention, in purified form, was determined as a half-life compared to the half-life of the hexosaminidase of SEQ ID NO: 3 using the methods described above (37° C. for 7 days in 80 vol % Persil® ProClean® Power Liquid 2in1 detergent). The results are expressed as a half-life improvement factor, HIF, compared to the half-life of SEQ ID NO: 3, in Table 4 below.

TABLE 4
Half-life improvement factor, HIF, of hexosaminidase variants

| Substitutions compared to | HIF compared to |
|---|---|
| SEQ ID NO: 3 | SEQ ID NO: 3 |
| V106N D111R T120V E150N | 14.75 |
| D111R T120V E150N | 19.94 |
| V106N T120V E150N D171F I278V | 24.39 |
| D111R T120V E150N D171F I278V | 20.41 |

### Example 5: Thermostability of Purified Hexosaminidase Polypeptides

The thermostability of the wildtype hexosaminidase from Terribacillus saccharophilus (SEQ ID NO: 1) and three variants thereof (SEQ ID NOs: 2, 3 and 4) was determined by nDSF using the method described above, in a model liquid detergent with the following composition:

TABLE 5
Composition of liquid model detergent

| | Content of active |
|---|---|
| Ingredient | component (% w/w) |
| LAS (linear alkylbenzene sulfonate) | 12 |
| AEO (alcohol ethoxylate) | 11 |
| AEOS (alcohol ether sulfate), SLES (sodium | 5 |
| lauryl ether sulfate) | |
| MPG (propane-1,2-diol) | 6 |
| Ethanol | 3 |
| TEA (2,2′,2″-nitrilotriethanol) | 3 |
| Coco fatty acid | 2.75 |
| Soy fatty acid | 2.75 |
| Glycerol | 2 |
| Sodium hydroxide (solid crystals) | 2 |
| Sodium citrate | 2 |
| Sodium formate | 1 |
| DTMPA (diethylenetriamine penta(methylene | 0.2 |
| phosphonic acid) | |
| PCA (copoly(acrylic acid/maleic acid), | 0.2 |
| sodium salt) | |

The results, expressed as a melting temperature (Tm), in detergent concentrations of 0, 2, 50 and 250 g/L, are provided in Table 6 below, where “-” indicates that the wildtype hexosaminidase of SEQ ID NO: 1 is unstable, so that a Tm value could not be determined in the higher detergent concentrations.

TABLE 6
Melting temperature, Tm, of hexosaminidase wildtype and variants

| | Tm ° C., | Tm ° C., | Tm ° C., | Tm ° C., |
|---|---|---|---|---|
| Hexosa- | 0 g/L | 2 g/L | 50 g/L | 250 g/L |
| minidase | detergent | detergent | detergent | detergent |
| SEQ ID NO: 1 | 50.5 | 40.9 | — | — |
| SEQ ID NO: 2 | 82.2 | 81.4 | 79.9 | 78.6 |
| SEQ ID NO: 3 | 84.8 | 83.6 | 82.4 | 81.5 |
| SEQ ID NO: 4 | 74.8 | 74.8 | 72.8 | 70.0 |

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1. A variant of the hexosaminidase polypeptide of SEQ ID NO: 1, wherein the variant comprises, compared to SEQ ID NO: 1:
a substitution at one or more positions corresponding to positions 163, 227, 252 and 309 of SEQ ID NO: 1; and
a substitution at one or more positions corresponding to positions 111, 120, 106, 124, 127, 150, 170, 171, 178, 199, 208, 254, 255 and 278 of SEQ ID NO: 1;
wherein the variant has hexosaminidase activity and has at least 60% sequence identity, but less than 100% sequence identity, to the polypeptide of SEQ ID NO: 1, SEQ ID NO:
2 or SEQ ID NO: 3.

2. The hexosaminidase variant of claim 1, wherein the variant comprises, compared to SEQ ID NO: 1,
two, three or four substitutions selected from the group consisting of S163P, N227T, N252P and K309E; and
at least one substitution selected from the group consisting of D111R, T120V, V106M, V106N, Y124I, Y124K, Y124L, R127P, E150N, L170G, D171F, D171H, Q178K, S199W, S208W, F254I, T255D and I278V.

3. The hexosaminidase variant of claim 1, wherein the variant further comprises, compared to SEQ ID NO: 1, at least one substitution selected from the group consisting of H15Y, S186R, S225G, E232D, G235W, N260Q, H272V, S279D, Y281P, K308Q and K312Q.

4. The hexosaminidase variant of claim 1, wherein the variant comprises, compared to SEQ ID NO: 1, at least one substitution selected from the group consisting of D111R, T120V, V106M/N, E150N, D171F and I278V; and wherein the variant has at least 80% sequence identity, to SEQ ID NO: 2 or SEQ ID NO: 3.

5. The hexosaminidase variant of claim 1, wherein the variant comprises, compared to SEQ ID NO: 2, at least one of the following substitutions or sets of substitutions:
D111R
D111R+T120V
D111R+T120V+E150N
D111R+T120V+E150N+D171F+I278V
D111R+T120V+T255D
D111R+T120V+Y124I
D111R+T120V+Y124K+Q178K
D111R+T120V+Y124L+Q178K
D111R+Y124K+D171FD171F+S208W+N267T
D171F+T255D
D171H
I278V
K308E
N267T
R127P
T120V+D171F
T120V+D171F+N267T
T120V+S208W+N267T
T120V+T255D
T120V+T255D+N267T
T120V+Y124K+Q178K
V106M+D111R+T120V+D171F
V106M+Q178K+T255D
V106M+T120V+D171F+Q178K
V106M+T120V+Y124I+D171F
V106M+T120V+Y124I+Q178K
V106M+T120V+Y124K+D171F
V106M+T120V+Y124L+Q178K
V106N+D111R+T120V+E150N
V106N+S208W+N267T
V106N+T120V+D171F
V106N+T120V+D171F+S208W
V106N+T120V+E150N+D171F+I278V
V106N+T120V+N267T.

6. The hexosaminidase variant of claim 1, wherein the variant comprises, compared to SEQ ID NO: 3, at least one of the following substitutions or sets of substitutions:
L170G
E150N+F254I
F254I
V106N
D111R+T120V
D111R+T120V+E150N
D111R+T120V+E150N+D171F+I278V
D111R+T120V+T255D
D111R+T120V+Y124I
D111R+T120V+Y124K+Q178K
D111R+T120V+Y124L+Q178K
D111R+Y124K+D171F
D171F+S208W+N267T
D171F+T255D
D171H
I278V
K308E
N267T
R127P
T120V+D171F
T120V+D171F+N267T
T120V+S208W+N267T
T120V+T255D
T120V+T255D+N267T
T120V+Y124K+Q178K
V106M+D111R+T120V+D171F
V106M+Q178K+T255D
V106M+T120V+D171F+Q178K
V106M+T120V+Y124I+D171F
V106M+T120V+Y124I+Q178K
V106M+T120V+Y124K+D171F
V106M+T120V+Y124L+Q178K
V106N+D111R+T120V+E150N
V106N+S208W+N267T
V106N+T120V+D171F
V106N+T120V+D171F+S208W
V106N+T120V+E150N+D171F+I278V
V106N+T120V+N267T.

7. The hexosaminidase variant of claim 1, wherein the variant comprises one of the following sets of substitutions compared to SEQ ID NO: 1:
Q3I+H15Y+A49W+N59E+D111R+T120V+S163P+S186R+S225G+N227T+E232D+G235W+N252P+N260Q+H272V+S279D+Y281P+K308Q+K309E+K312Q
Q3I+H15Y+A49W+N59E+D111R+T120V+E150N+S163P+S186R+S225G+N227T+E232D+G235W+N252P+N260Q+H272V+S279D+Y281P+K308Q+K309E+K312Q
Q3I+H15Y+A49W+N59E+D111R+T120V+E150N+S163P+D171F+S186R+S225G+N227T+E232D+G235W+N252P+N260Q+H272V+I278V+S279D+Y281P+K308Q+K309E+K312Q
Q3I+H15Y+A49W+N59E+D111R+T120V+S163P+S186R+S225G+N227T+E232D+G235W+N252P+T255D+N260Q+H272V+S279D+Y281P+K308Q+K309E+K312Q
Q3I+H15Y+A49W+N59E+D111R+T120V+Y124I+S163P+S186R+S225G+N227T+E232D+G235W+N252P+N260Q+H272V+S279D+Y281P+K308Q+K309E+K312Q
Q3I+H15Y+A49W+N59E+D111R+T120V+Y124K+S163P+Q178K+S186R+S225G+N227T+E232D+G235W+N252P+N260Q+H272V+S279D+Y281P+K308Q+K309E+K312Q
Q3I+H15Y+A49W+N59E+D111R+T120V+Y124L+S163P+Q178K+S186R+S225G+N227T+E232D+G235W+N252P+N260Q+H272V+S279D+Y281P+K308Q+K309E+K312Q
Q3I+H15Y+A49W+N59E+D111R+Y124K+S163P+D171F+S186R+S225G+N227T+E232D+G235W+N252P+N260Q+H272V+S279D+Y281P+K308Q+K309E+K312Q
Q3I+H15Y+A49W+N59E+S163P+D171F+S186R+S208W+S225G+N227T+E232D+G235W+N252P+N260Q+N267T+H272V+S279D+Y281P+K308Q+K309E+K312Q
Q3I+H15Y+A49W+N59E+S163P+D171F+S186R+S225G+N227T+E232D+G235W+N252P+T255D+N260Q+H272V+S279D+Y281P+K308Q+K309E+K312Q
Q3I+H15Y+A49W+N59E+S163P+D171H+S186R+S225G+N227T+E232D+G235W+N252P+N260Q+H272V+S279D+Y281P+K308Q+K309E+K312Q
Q3I+H15Y+A49W+N59E+S163P+S186R+S225G+N227T+E232D+G235W+N252P+N260Q+H272V+I278V+S279D+Y281P+K308Q+K309E+K312Q
Q3I+H15Y+A49W+N59E+S163P+S186R+S225G+N227T+E232D+G235W+N252P+N260Q+H272V+S279D+Y281P+K308E+K309E+K312Q
Q3I+H15Y+A49W+N59E+S163P+S186R+S225G+N227T+E232D+G235W+N252P+N260Q+N267T+H272V+S279D+Y281P+K308Q+K309E+K312Q
Q3I+H15Y+A49W+N59E+R127P+S163P+S186R+S225G+N227T+E232D+G235W+N252P+N260Q+H272V+S279D+Y281P+K308Q+K309E+K312Q
Q3I+H15Y+A49W+N59E+T120V+S163P+D171F+S186R+S225G+N227T+E232D+G235W+N252P+N260Q+H272V+S279D+Y281P+K308Q+K309E+K312Q
Q3I+H15Y+A49W+N59E+T120V+S163P+D171F+S186R+S225G+N227T+E232D+G235W+N252P+N260Q+N267T+H272V+S279D+Y281P+K308Q+K309E+K312Q
Q3I+H15Y+A49W+N59E+T120V+S163P+S186R+S208W+S225G+N227T+E232D+G235W+N252P+N260Q+N267T+H272V+S279D+Y281P+K308Q+K309E+K312Q
Q3I+H15Y+A49W+N59E+T120V+S163P+S186R+S225G+N227T+E232D+G235W+N252P+T255D+N260Q+H272V+S279D+Y281P+K308Q+K309E+K312Q
Q3I+H15Y+A49W+N59E+T120V+S163P+S186R+S225G+N227T+E232D+G235W+N252P+T255D+N260Q+N267T+H272V+S279D+Y281P+K308Q+K309E+K312Q
Q3I+H15Y+A49W+N59E+T120V+Y124K+S163P+Q178K+S186R+S225G+N227T+E232D+G235W+N252P+N260Q+H272V+S279D+Y281P+K308Q+K309E+K312Q
Q3I+H15Y+A49W+N59E+V106M+D111R+T120V+S163P+D171F+S186R+S225G+N227T+E232D+G235W+N252P+N260Q+H272V+S279D+Y281P+K308Q+K309E+K312Q
Q3I+H15Y+A49W+N59E+V106M+S163P+Q178K+S186R+S225G+N227T+E232D+G235W+N252P+T255D+N260Q+H272V+S279D+Y281P+K308Q+K309E+K312Q
Q3I+H15Y+A49W+N59E+V106M+T120V+S163P+D171F+Q178K+S186R+S225G+N227T+E232D+G235W+N252P+N260Q+H272V+S279D+Y281P+K308Q+K309E+K312Q
Q3I+H15Y+A49W+N59E+V106M+T120V+Y124I+S163P+D171F+S186R+S225G+N227T+E232D+G235W+N252P+N260Q+H272V+S279D+Y281P+K308Q+K309E+K312Q
Q3I+H15Y+A49W+N59E+V106M+T120V+Y124I+S163P+Q178K+S186R+S225G+N227T+E232D+G235W+N252P+N260Q+H272V+S279D+Y281P+K308Q+K309E+K312Q
Q3I+H15Y+A49W+N59E+V106M+T120V+Y124K+S163P+D171F+S186R+S225G+N227T+E232D+G235W+N252P+N260Q+H272V+S279D+Y281P+K308Q+K309E+K312Q
Q3I+H15Y+A49W+N59E+V106M+T120V+Y124L+S163P+Q178K+S186R+S225G+N227T+E232D+G235W+N252P+N260Q+H272V+S279D+Y281P+K308Q+K309E+K312Q
Q3I+H15Y+A49W+N59E+V106N+D111R+T120V+E150N+S163P+S186R+S225G+N227T+E232D+G235W+N252P+N260Q+H272V+S279D+Y281P+K308Q+K309E+K312Q
Q3I+H15Y+A49W+N59E+V106N+S163P+S186R+S208W+S225G+N227T+E232D+G235W+N252P+N260Q+N267T+H272V+S279D+Y281P+K308Q+K309E+K312Q
Q3I+H15Y+A49W+N59E+V106N+T120V+S163P+D171F+S186R+S225G+N227T+E232D+G235W+N252P+N260Q+H272V+S279D+Y281P+K308Q+K309E+K312Q
Q3I+H15Y+A49W+N59E+V106N+T120V+S163P+D171F+S186R+S208W+S225G+N227T+E232D+G235W+N252P+N260Q+H272V+S279D+Y281P+K308Q+K309E+K312Q
Q3I+H15Y+A49W+N59E+V106N+T120V+E150N+S163P+D171F+S186R+S225G+N227T+E232D+G235W+N252P+N260Q+H272V+I278V+S279D+Y281P+K308Q+K309E+K312Q
Q3I+H15Y+A49W+N59E+V106N+T120V+S163P+S186R+S225G+N227T+E232D+G235W+N252P+N260Q+N267T+H272V+S279D+Y281P+K308Q+K309E+K312Q
Q3F+H15Y+V140I+S163P+L170G+S186R+Q215K+S225G+N227T+E232D+G235W+N252P+N260Q+N267T+H272V+S279D+Y281P+K308Q+K309E+K312Q
Q3F+H15Y+V140I+E150N+S163P+S186R+Q215K+S225G+N227T+E232D+G235W+N252P+F254I+N260Q+N267T+H272V+S279D+Y281P+K308Q+K309E+K312Q
Q3F+H15Y+V140I+S163P+S186R+Q215K+S225G+N227T+E232D+G235W+N252P+F254I+N260Q+N267T+H272V+S279D+Y281P+K308Q+K309E+K312Q
Q3F+H15Y+V106N+V140I+S163P+S186R+Q215K+S225G+N227T+E232D+G235W+N252P+N260Q+N267T+H272V+S279D+Y281P+K308Q+K309E+K312Q
Q3F+H15Y+D111R+V140I+S163P+S186R+Q215K+S225G+N227T+E232D+G235W+N252P+N260Q+N267T+H272V+S279D+Y281P+K308Q+K309E+K312Q
Q3F+H15Y+D111R+V140I+E150N+S163P+S186R+Q215K+S225G+N227T+E232D+G235W+N252P+N260Q+N267T+H272V+S279D+Y281P+K308Q+K309E+K312Q
Q3F+H15Y+D111R+V140I+E150N+S163P+L170G+S186R+Q215K+S225G+N227T+E232D+G235W+N252P+N260Q+N267T+H272V+S279D+Y281P+K308Q+K309E+K312Q
Q3F+H15Y+D111R+V140I+E150N+S163P+L170G+S186R+Q215K+S225G+N227T+E232D+G235W+N252P+N260Q+N267T+H272V+I278V+S279D+Y281P+K308Q+K309E+K312Q
Q3F+H15Y+D111R+V140I+E150N+S163P+S186R+S199W+Q215K+S225G+N227T+E232D+G235W+N252P+N260Q+N267T+H272V+I278V+S279D+Y281P+K308Q+K309E+K312Q
Q3F+H15Y+D111R+V140I+S163P+S186R+Q215K+S225G+N227T+E232D+G235W+N252P+N260Q+N267T+H272V+I278V+S279D+Y281P+K308Q+K309E+K312Q
Q3F+H15Y+D111R+V140I+S163P+L170G+S186R+Q215K+S225G+N227T+E232D+G235W+N252P+N260Q+N267T+H272V+S279D+Y281P+K308Q+K309E+K312Q
Q3F+H15Y+D111R+V140I+S163P+L170G+S186R+Q215K+S225G+N227T+E232D+G235W+N252P+N260Q+N267T+H272V+I278V+S279D+Y281P+K308Q+K309E+K312Q
Q3F+H15Y+D111R+V140I+S163P+L170G+S186R+S199W+Q215K+S225G+N227T+E232D+G235W+N252P+N260Q+N267T+H272V+S279D+Y281P+K308Q+K309E+K312Q
Q3F+H15Y+V140I+E150N+S163P+S186R+Q215K+S225G+N227T+E232D+G235W+N252P+N260Q+N267T+H272V+S279D+Y281P+K308Q+K309E+K312Q
Q3F+H15Y+V140I+E150N+S163P+S186R+Q215K+S225G+N227T+E232D+G235W+N252P+N260Q+N267T+H272V+I278V+S279D+Y281P+K308Q+K309E+K312Q
Q3F+H15Y+V140I+E150N+S163P+L170G+S186R+Q215K+S225G+N227T+E232D+G235W+N252P+N260Q+N267T+H272V+S279D+Y281P+K308Q+K309E+K312Q
Q3F+H15Y+V140I+E150N+S163P+L170G+S186R+Q215K+S225G+N227T+E232D+G235W+N252P+N260Q+N267T+H272V+I278V+S279D+Y281P+K308Q+K309E+K312Q
Q3F+H15Y+V140I+E150N+S163P+S186R+S199W+Q215K+S225G+N227T+E232D+G235W+N252P+N260Q+N267T+H272V+S279D+Y281P+K308Q+K309E+K312Q
Q3F+H15Y+V140I+S163P+S186R+Q215K+S225G+N227T+E232D+G235W+N252P+N260Q+N267T+H272V+I278V+S279D+Y281P+K308Q+K309E+K312Q
Q3F+H15Y+V140I+S163P+L170G+S186R+Q215K+S225G+N227T+E232D+G235W+N252P+N260Q+N267T+H272V+S279D+Y281P+K308Q+K309E+K312Q
Q3F+H15Y+V140I+S163P+L170G+S186R+Q215K+S225G+N227T+E232D+G235W+N252P+N260Q+N267T+H272V+I278V+S279D+Y281P+K308Q+K309E+K312Q
Q3F+H15Y+V140I+S163P+L170G+S186R+S199W+Q215K+S225G+N227T+E232D+G235W+N252P+N260Q+N267T+H272V+S279D+Y281P+K308Q+K309E+K312Q
Q3F+H15Y+V140I+S163P+L170G+S186R+S199W+Q215K+S225G+N227T+E232D+G235W+N252P+N260Q+N267T+H272V+I278V+S279D+Y281P+K308Q+K309E+K312Q
Q3F+H15Y+V140I+S163P+S186R+S199W+Q215K+S225G+N227T+E232D+G235W+N252P+N260Q+N267T+H272V+I278V+S279D+Y281P+K308Q+K309E+K312Q.

8. The hexosaminidase variant of claim 1, wherein the variant has at least 80% sequence identity, to SEQ ID NO: 2 or SEQ ID NO: 3.

9. The hexosaminidase variant of claim 1, wherein the variant has improved stability compared to the polypeptide of SEQ ID NO: 1, SEQ ID NO: 2 or SEQ ID NO: 3, or to a parent polypeptide having the same amino acid sequence as the variant without said substitutions.

10. A detergent composition comprising the hexosaminidase variant of claim 1 and at least one detergent adjunct ingredient.

11. (canceled)

12. A method of cleaning an item, comprising exposing the item to a wash liquor comprising the detergent composition of claim 10.

13. A method for laundering a textile, comprising:
a) exposing the textile to a wash liquor comprising the detergent composition of claim 10;
b) completing at least one wash cycle; and optionally
c) rinsing the item.

14. A polynucleotide encoding the hexosaminidase variant of claim 1, a nucleic acid construct or expression vector comprising the polynucleotide, or a recombinant host cell transformed with the polynucleotide.

15. A method of producing a hexosaminidase variant, comprising:
a) cultivating the recombinant host cell of claim 14 under conditions suitable for expression of the variant; and
b) recovering the variant.
